(19) United States
(12) Patent Application Publication
(10) Pub. No.: US 2004/0152741 A1
(43) Pub. Date: Aug. 5, 2004

(54) Arylglycine derivatives and their use as glycine transport inhibitors

(75) Inventors: Methvin Isaac, Etobicoke (CA); Tao Xin, Woodbridge (CA); Louise Edwards, Mississauga (CA); Leah Begleiter, Toronto (CA); Tomislav Stefanac, Burlington (CA); Anne O'Brien, Toronto (CA); Kathleen Da Silva-Turcot, Toronto (CA); Jalaj Arora, Milton (CA); Shawn Maddaford, Mississauga (CA); Abdelmalik Slassi, Mississauga (CA)

Correspondence Address: JOHN W. RYAN, C/O DECHERT LLP, PRINCETON PIKE CORPORATION CENTER, P.O. BOX 5218, PRINCETON, NJ, 08543-5218, US

(73) Assignee: NPS Allelix Corporation, Mississauga (CA)

(21) Appl. No.: 10/657,815

(22) Filed: Sep. 8, 2003

Related U.S. Application Data
(60) Provisional application No. 60/409,421, filed on Sep. 9, 2002.

(51) Int. Cl. C07D 213/75; A61K 31/44; A61K 31/40; C07D 207/34; A61K 31/17

(52) U.S. Cl. 514/352; 514/426; 514/586; 514/597; 546/305; 548/557; 564/21; 564/48

(57) ABSTRACT

The present invention relates to compounds of Formula 1,

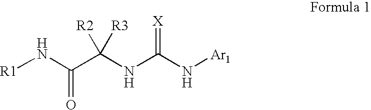

and salts solvates and hydrates thereof. The invention further relates to pharmaceutical compositions containing said compounds and methods of treating neurological, neuropsychiatric, and gastrointestinal disorders using said compounds.

This application claims priority to U.S. Provisional Application No. 60/409,421 filed Sep. 9, 2002.

The present invention relates to a class of compounds, to pharmaceutical compositions containing them and to methods of treating neurological and neuropsychiatric and gastrointestinal disorders using such compounds.

### BACKGROUND OF THE INVENTION

Synaptic transmission is a complex form of intercellular communication that involves a considerable array of specialized structures in both the pre- and post-synaptic terminal and surrounding glial cells (Kanner and Schuldiner, CRC Critical Reviews in Biochemistry, 22, 1987:1032). Transporters sequester neurotransmitters from the synapse, thereby regulating the concentration of neurotransmitters in the synapse, and their duration therein, which together influence the magnitude of synaptic transmission. Further, by preventing the spread of neurotransmitter to neighbouring synapses, transporters maintain the fidelity of synaptic transmission. Lastly, by sequestering released neurotransmitter into the presynaptic terminal, transporters allow for neurotransmitter re-utilization.

Neurotransmitter transport is dependent upon extracellular sodium and the voltage difference across the membrane. Under conditions of intense neuronal firing, for example, during a seizure, transporters can function in reverse, releasing neurotransmitter in a calcium-independent non-exocytotic manner (Attwell et al., Neuron, 11, 1993:401-407). Pharmacologic modulation of neurotransmitter transporters thus provides a means for modifying synaptic activity, which provides useful therapy for the treatment of neurological and psychiatric disturbances.

The amino acid glycine is a major neurotransmitter in the mammalian central nervous system, functioning at both inhibitory and excitatory synapses. By nervous system, both the central and peripheral portions of the nervous system are intended. These distinct functions of glycine are mediated by two different types of receptor, the glycine receptor and the NMDA receptor, each of which is associated with a different class of glycine transporter. The inhibitory actions of glycine are mediated by glycine receptors that are sensitive to the convulsant alkaloid strychnine, and are thus referred to as “strychnine-sensitive”, Such receptors contain an intrinsic chloride channel that is opened upon binding of glycine to the receptor; by increasing chloride conductance, the threshold for firing of an action potential is increased. Strychnine-sensitive glycine receptors are found predominantly in the spinal cord and brainstem, and pharmacological agents that enhance the activation of such receptors will thus increase inhibitory neurotransmission in these regions.

Glycine also functions in excitatory transmission by modulating the actions of glutamate, the major excitatory neurotransmitter in the central nervous system (Johnson and Ascher, Nature, 325, 1987:529-531; Fletcher et al., Glycine Transmission, Otterson and Storm-Mathisen, eds., 1990:193-219). Specifically, glycine is thought to be an obligatory co-agonist at the class of glutamate receptor termed N-methyl-D-aspartate (NMDA) receptor. Activation of NMDA receptors increases sodium and calcium conductance, which depolarizes the neuron, thereby increasing the likelihood that it will fire an action potential.

NMDA receptors in the hippocampal region of the brain play an important role in a model of synaptic plasticity known as long-term potentiation (LTP), which is integral in certain types of learning and memory (Hebb, D. O (1949) The Organization of Behavior, Wiley, N.Y.; Bliss and Collingridge (1993) Nature 361: 31-39; Morris et al. (1986) Nature 319: 774-776). Enhanced expression of selected NMDA receptor sub-units in transgenic mice results in increased NMDA-receptor-mediated currents, enhanced LTP, and better performance in some tests of learning and memory (Tang et al. (1999) Nature 401: 63).

Conversely, decreased expression of selected NMDA receptor sub-units in transgenic mice produces behaviors similar to pharmacologically-induced animal models of schizophrenia, including increased locomotion, increased stereotypy, and deficits in social/sexual interactions (Mohn et al. (1999) Cell 98:427-436). These aberrant behaviors can be ameliorated using the antipsychotics haloperidol and clozapine.

NMDA receptors are widely distributed throughout the brain, with a particularly high density in the cerebral cortex and hippocampal formation.

Molecular cloning has revealed the existence of two classes of glycine transporters in mammalian brains, termed GlyT-1 and GlyT-2. GlyT-1 is found throughout the brain and spinal cord, and it has been suggested that its distribution corresponds to that of glutamatergic pathways and NMDA receptors (Smith, et al., Neuron, 8, 1992:927-935). Molecular cloning has further revealed the existence of four variants of GlyT-1, termed GlyT-1a, GlyT-1b, GlyT-1c and GlyT-1d. Two of these variants (1a and 1b) are found in rodents, each of which displays a unique distribution in the brain and peripheral tissues (Borowsky et al., Neuron, 10, 1993:851-863; Adams et al., J. Neuroscience, 15, 1995:2524-2532). The third variant, 1c, has only been detected in human tissues (Kim, et al., Molecular Pharmacology, 45, 1994:608-617). The fourth variant has been detected in human tissues (see U.S. Pat. No. 6,008,015). These variants arise by differential splicing and exon usage, and differ in their N-terminal regions. GlyT-2, is found predominantly in the brain stem and spinal cord, and its distribution corresponds closely to that of strychnine-sensitive glycine receptors (Liu et al., J. Biological Chemistry, 268, 1993:22802-22808; Jursky and Nelson, J. Neurochemistry, 64, 1995:1026-1033). Another distinguishing feature of glycine transport mediated by GlyT-2 is that it is not inhibited by sarcosine as is the case for glycine transport mediated by GlyT-1. These data are consistent with the view that, by regulating the synaptic levels of glycine, GlyT-1 and GlyT-2 selectively influence the activity of NMDA receptors and strychnine-sensitive glycine receptors, respectively.

Compounds which inhibit or activate glycine transporters would thus be expected to alter receptor function by modifying glycine concentrations in the synapse and, thus, provide therapeutic benefits in a variety of disease states.

For example, compounds which inhibit GlyT-1 mediated glycine transport may increase glycine concentrations at NMDA receptors, which receptors are located in the forebrain, among other locations. This concentration increase could perhaps elevate the activity of NMDA receptors, thereby possibly alleviating symptoms of schizophrenia and enhancing cognitive function. Alternatively, compounds that interact directly with the glycine receptor component of the NMDA receptor can have the same or similar effects as increasing or decreasing the availability of extracellular glycine caused by inhibiting or enhancing GlyT-1 activity, respectively. See, for example, Pitkänen et al., Eur. J. Pharmacol., 253, 125-129 (1994); Thiels et al., Neuroscience, 46, 501-509 (1992); and Kretschmer and Schmidt, J. Neurosci., 16, 1561-1569 (1996).

### SUMMARY OF THE INVENTION

According to one aspect of the invention, there are provided compounds of Formula I:

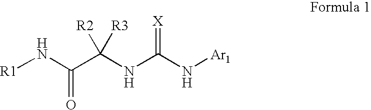

wherein:

R₁ is selected from cycloalkyl, heterocycloalkyl, aryl and heteroaryl;

wherein R₁ is optionally substituted with one or more substituents Rₐ, wherein Rₐ may be independently selected from the group consisting of alkyl, halo, haloalkyl, nitro, alkenyl, alkynyl, alkoxy, —(R₇)ₙNR₈R₉ (wherein R₇ is selected from alkyl, alkoxy, and oxyalkyl, R₈ and R₉ can be independently selected from H, and alkyl, or R₈ and R₉ can join such that NR₈R₉ form a 5 or 6 member heterocyclic ring, and n is selected from 0, and 1), and the substituent Rₐ is optionally further substituted with one or more substituents selected from the group consisting of alkyl, alkoxy, halo, cyano, alkanoyl, haloalkyl, thioalkyl, nitro, and —(R₇)ₙNR₈R₉ wherein R₇, R₈, and R₉, and n are as defined above.

R₂ and R₃ are

a) independently selected from the group consisting of H, alkyl, haloalkyl, aralkyl optionally substituted aryl, optionally substituted heteroaryl and optionally substituted, saturated or unsaturated, 5-or 6-membered, homocyclic or heterocyclic rings wherein the optional substituent may be selected from the group consisting of H, alkyl, alkoxy, and halo;

or

b) join together to form a 3, 4, 5, 6 or 7 member spirocyclic ring;

X is selected from the group consisting of O, S, NH and NCN;

Ar₁ is phenyl and is optionally substituted with one or more substituents R_{b},

wherein the substituents R_{b} are independently selected from the group consisting of alkyl, alkoxy, nitro, halo, haloalkoxy, —(R₇)ₙNR₈R₉ —S(O)₂NR₁₀R₁₁, and —O—(CH₂)ₘNR₁₀R₁₁ (wherein R₇ is selected from alkyl, alkoxy, and oxyalkyl, R₈ and R₉ can be independently selected from H, and alkyl, or R₈ and R₉ can join together such that NR₈R₉ form a 5 or 6 member heterocyclic ring, and n is selected from 0, 1, 2, 3, 4 and 5 and R₁₀ and R₁₁ can be independently selected from H, or alkyl, or R₁₀ and R₁₁ can join together such that NR₁₀R₁₁ form a 5 or 6 member heterocyclic ring and m is selected from 1, 2, 3, 4, and 5 ) and;

the substituent R_{b} is optionally further substituted with one or more substituents selected from the group consisting of alkyl, alkoxy, halo, cyano, alkanoyl, haloalkyl, thioalkyl, nitro, —(R₇)ₙNR₈R₉ (wherein R₇, R₈, R₉ and n are as described above), with the proviso that Ar₁ does not have a substituent at the 2-position selected from the following groups, nitro haloalkyl, cyano, —C(O)R₁₂ —C(O)OR₁₂, —C(O)NR₁₂R₁₃, —S(O)R₁₂, —S(O)₂R₁₂, and —S(O)₂NR₁₂R₁₃ (wherein R₁₂ and R₁₃ are independently selected from H and alkyl),

and a second proviso that Ar₁ does not have an alkanoyl substituent at the 4 position,

and a salt solvate of hydrate thereof.

It has been found that compounds of Formula I inhibit glycine transport via GlyT-1, or are precursors (for example, pro-drugs) of such compounds. GlyT-1 transport inhibitors may be useful in the treatment of schizophrenia, as well as other CNS-related disorders such as cognitive dysfunction, dementia (including that related to Alzheimer's disease), attention deficit disorder, depression and intestinal disorders.

According to another aspect of the invention, there is provided a pharmaceutical composition comprising a compound of Formula I in an amount effective to inhibit glycine transport, and a pharmaceutically acceptable carrier.

In another aspect of the invention, there are provided compositions containing compounds of Formula 1 in amounts suitable for pharmaceutical use to treat medical conditions for which a glycine transport inhibitor is indicated. Preferred are those compositions containing compounds useful in the treatment of medical conditions for which GlyT-1-mediated inhibition of glycine transport is needed, such as the treatment of schizophrenia, cognitive dysfunction, or Alzheimer's.

Definitions

The term “aryl” as used herein means a 5, 6, 7, 8, 9 or 10 member monocyclic, bicyclic, or benzo-fused aromatic group such as phenyl, naphthyl, indanyl, tetrahydronaphthyl, dihydronaphthyl, indenyl and the like.

The term “heteroatom” as used herein means a non-carbon atom such as S, N, O and the like.

The term “heteroaryl” as used herein means an aryl group containing 1, 2 or 3 heteroatoms selected from N, O and S with the proviso that no two like heteroatoms are adjacent unless both are N, and includes such compounds as pyridyl, furyl, thienyl, pyrimidinyl, pyrollyl, imidazolyl, triazolyl, oxazolyl, thiazolyl, oxadiazolyl, thiadiazolyl, quinolinyl, quinoxylinyl, quinazolinyl, pyrazinyl, pyrimidinyl, indolyl, indazolyl, azaindazolyl, isoquinolinyl, and the like.

The term “alkyl” as used herein means straight- and branched-chain alkyl radicals containing from 1, 2, 3, 4, 5 or 6 carbon atoms and includes methyl, ethyl, proplyl, isopropyl, butyl, s-butyl, t-butyl n-pentyl, l-pentyl, neopentyl, hexyl, and the like.

The term “cycloalkyl” as used herein means a carbocyclic ring containing 3, 4, 5, 6, 7 or 8 carbon atoms and includes cyclopropyl, cyclopentyl, cyclohexyl cycloheptyl, cyclooctyl and the like.

The term “heterocycloalkyl” as used herein means a 3, 4, 5, 6, 7 or 8-membered ring containing one or two heteroatoms selected from the group consisting of N, S, and O and includes piperidinyl, piperazinyl, tetrahydopyran, tetrahydrothiopyran, morpholine thiomorpholine, tetrahydrofuran, tetrahydrothiophene, pyrolidine, and the like.

The term “alkoxy” as used herein means straight- and branched-chain alkoxy radicals containing 1, 2, 3, 4, 5 or 6 carbon atoms and includes methoxy, ethoxy, propoxy, isopropoxy, butoxy, tertbutoxy, pentoxy, hexyloxy and the like.

The term “aralkyl” as used herein means an alkyl radical as previously described substituted with an aryl group as previously described and includes benzyl, phenethyl and the like.

The term “aralkoxy” as used herein means an alkoxy radical substituted with an aryl group such as benzyloxy, phenethyloxy and the like.

The term “aryloxy” as used herein means an aryl substituted oxy radical such as phenoxy.

The terms “alkylene”, “alkenylene” and “alkynylene” as used herein means straight- and branched-chain bivalent radicals containing 1, 2, 3, 4, 5 or 6 carbon atoms, such as methylene, ethylene, 2-butenyl, vinyl, propenylene and ethynylene.

The term “alkanoyl” as used herein means straight- and branched-chain radicals containing 1, 2, 3, 4, 5 or 6 carbon atoms and includes acetyl, ethanoyl, propionyl, butanoyl, pentanoyl, hexanoyl and the like.

The term “halo” as used herein means halogen and includes fluoro, chloro, bromo and iodo.

The term “haloalkyl” as used herein means a straight or branched chain alkyl radical of 1, 2, 3, 4, 5 or 6 carbons with one or more halogen susbtituents such as trifluoromethyl, bromoethyl, chloromethyl, chlorohexyl and the like.

The term “thioalkyl” as used herein means straight- and branched-chain alkyl containing 1, 2, 3, 4, 5 or 6 carbons bonded through a sulfur radical and includes thiomethyl (CH₃S—), thioethyl, thiopropyl thiobutyl, thiophenyl, thiohexyl and the like.

The term “sulfonamido” as used herein means sulfonamide radicals where the nitrogen may be unsubstituted or substituted or a member of a ring and includes —S(O)₂NRR, wherein R can be H, alkyl, alkoxy, cycloalkyl, aryl, and the like or the two R groups may join together such that NRR forms a ring.

The term “SPE tube” as used herein refers to a solid phase extraction tube. These may be commercially prepared disposable tube filled with Silica gel for carrying out chromatography. Such tubes can be purchased from Varian and Supelco.

The term “pharmaceutically acceptable salt” means an acid addition salt, which is compatible with the treatment of patients.

A “pharmaceutically aceptable addition salt” is any non-toxic organic or inorganic acid addition salt of the base compounds represented by Formula 1 or any of Formula 1's intermediates. Illustrative inorganic acids which form suitable salts include hydrochloric, hydrobromic, sulfuric, and phosphoric acid and acid metal salts such as sodium monohydrogen, orthophosphate and potassium hydrogensulfate. Illustrative organic acids which form suitable salts include the mono-, di-, and tricarboxylic acids. Illustrative of such acids are for example acetic, glycolic, lactic, pyruvic, malonic, succinic, glutaric, fumaric, malic, tartaric, citric, ascorbic, maleic, hydromaleic, benzoic, hydroxybenzoic, phenylacetic, cinnamic, salicylic, 2-phenoxybenzoic, p-toluensulfonic acid and other sulfonic acids such as methanesulfonic acid and 2-hydroxyethanesulfonic acid. Either the mono- or di-acid salts can be formed and such salts can exist in either a hydrated, solvated, or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art.

The term “solvate” as used herein means a compound of Formula 1 wherein molecules of a suitable solvent are incorporated in a crystal lattice. A suitable solvent is physiologically tolerable at the dosage administered as the solvate. Examples of suitable solvents are ethanol and the like.

The term “stereoisomers” is a general term for all isomers of the individual molecules that differ only in the orientation of their atoms in space. It includes mirror image isomers (enantiomers), geometric isomers (cis/trans) and isomers of compounds with more than one chiral centre that are not mirror images of one another (diastereomers).

The term “treat” or “treating” means to alleviate symptoms, eliminate the causation of the symptoms, either on a temporary or permanent basis, or to prevent or slow the appearance of symptoms of the named disorder or condition.

The term “therapeutically acceptable carrier” means a non-toxic solvent, dispersant, excipient, adjuvant, or other material which is mixed with the active ingredient in order to permit the formation of a pharmaceutical composition, i.e., a dosage form capable of administration to the patient.

### DETAILED DESCRIPTION AND PREFERRED EMBODIMENTS

Compounds of the invention include compounds of Formula 1. Compounds of Formula 1 include those in which R₁ is selected from cycloalkyl, heterocycloalkyl, aryl and heteroaryl and R₁ is optionally substituted. In suitable embodiments of the invention R₁ is optionally substituted and is selected from naphthalene tetrahydronaphthalene or quinoline. In further embodiments of the invention R₁ is unsubstituted and selected from naphthalene tetrahydronaphthalene or quinoline.

In another suitable embodiment of the invention R₁ is optionally substituted pyridine. In further embodiments of the invention R₁ is 3-pyridyl optionally substituted with alkyl or haloalkyl groups. In still further embodiments of the invention the substituent is at the 6 position, for example 6-methyl-3-pryridyl or 6-trifluoromethyl-3-pyridyl.

In yet a further embodiment of the invention R₁ is optionally substituted phenyl wherein substituents Rₐ, are selected from alkyl, alkoxy, alkanoyl, halo, —(R₇)ₙNR₈R₉, (wherein R₇ is selected from alkyl, alkoxy, and oxyalkyl, R₈ and R₉ can be independently selected from H, and alkyl, or R₈ and R₉ can join together such that NR₈R₉ form a 5 or 6 member heterocyclic ring, and n is selected from 0 and 1), aryl, nitro, alkeny, haloalkyl, haloalkoxy, thioalkyl, cyano, and substituted or unsubstituted piperazinyl. In more particular embodiments, the substituents Rₐ are selected from the group consisting of methyl, Br, Cl, trifluromethyl, nitro, ipropyl, vinyl, methoxy, Et₂N-, trifluromethoxy, methythio, ethyl, phenyl, cyano, N-methylpiperazinyl.

In a further embodiment of the invention R₁ is selected from mono or di-substituted phenyl wherein the substituents, in order of preference, are located at the 3 and 4 positions>the 4 position>the 3 position>the 3 and 5 position>the 2 and 6 position>the 2 position>the 2 and 3 position. In another particular embodiment R₁ is 4-chlorophenyl, or 3-trifluoromethylphenyl. In another particular embodiment R₁ is 3,4-dimethylpheyl. In yet another particular embodiment R₁ is indanyl.

Another aspect of the invention includes compounds of Formula 1 wherein Ar₁ is optionally substituted phenyl. In a further embodiment of the invention Ar₁ is optionally substituted phenyl wherein the substituents R_{b} are selected from alkyl, alkoxy, nitro, halo, haloalkoxy, —(R₇)ₙNR₈R₉ (wherein R₇ is selected from alkyl, alkoxy, and oxyalkyl, R₈ and R₉ can be independently selected from H, and alkyl, or R₈ and R₉ can join together such that NR₈R₉ form a 5 or 6-member heterocyclic ring, and n is selected from 0, 1, 2, 3, 4 and 5) —S(O)₂NR₁₀R₁₁, and —O—(CH₂)ₘNR₁₀R₁₁ (wherein R₁₀ and R₁₁ can be independently selected from H, or alkyl, or R10 and R11 can join together such that NR₁₀R₁₁ form a 5 or 6-member heterocyclic ring and m is selected from 1, 2, 3, 4, and 5). The substituents R_{b} are optionally further substituted with one or more substituents selected from the group consisting of alkyl, alkoxy, halo, cyano, alkanoyl, haloalkyl, thioalkyl, nitro and —(R₇)ₙNR₈R₉, wherein R₇, R₈, R₉ and n are as defined above. Furthermore there is a proviso that, for the compounds of the present invention, Ar₁ does not have a substituent at the 2-position selected from the following groups, nitro haloalkyl, cyano, —C(O)R₁₂ —C(O)OR₁₂, —C(O)NR₁₂R₁₃, —S(O)R₁₂, —S(O)₂R₁₂, and —S(O)₂NR₁₂R₁₃ (wherein R₁₂ and R₁₃ are independently selected from H and alkyl), and a second proviso that Ar₁ does not have an alkanoyl substituent at the 4 position.

In another embodiment of the invention Ar₁ is mono or di-substituted phenyl wherein the substituents, in order of preference, are located at the 2 and 5 position>the 2 and 4 position>the 2 position>the 4 position>the 3 position, in order of preference.

In a further embodiment Ar₁ is substituted phenyl wherein the substituents are selected independently from alkyl, alkoxy, halo, alkanoyl, nitro, trifluromethyl and —(R₇)ₙNR₈R₉. In yet a further embodiment Ar₁ is di-substituted phenyl with the substituents selected from methoxy, nitro, F, Cl, ethoxy, trifluoromethyl, N-methylpiperidinyl, N′N-dimethylsulphonamide, (CH₃)₂NCH₂CH₂O—, and acetyl. In still a further embodiment of the invention R₄ is di-substituted phenyl with the substituents selected from nitro and MeO—. In a further embodiment Ar₁ is 2-methoxy-5-nitrophenyl.

In another embodiment of the invention R₂ and R₃ are independently selected from, H, alkyl, haloalkyl, aralkyl optionally substituted aryl, and optionally substituted heteroaryl, and optionally substituted, saturated or unsaturated 5, or 6-member homocyclic or heterocyclic rings.

In another suitable embodiment of the invention R₂ and R₃ are selected independently from H, phenyl, 3-thiophene, s-butyl, 3,4-difluorophenyl, cyclohexyl, 3-trifuoromethylphenyl, t-butyl, i-propyl, methyl, benzyl, trifuoromethyl. In yet another suitable embodiment R₂ is H and R₃ is selected from phenyl, 3-thiophene, s-butyl, 3,4-difluorophenyl, cyclohexyl, 3-trifuoromethylphenyl, t-butyl, i-propyl, methyl, benzyl, trifuoromethyl. In yet a further embodiment R₂ is H and R₃ is selected from phenyl and 3-thiophene. In still a further embodiment of the invention R₂ and R₃ together form a 3, 5 or 6-member spirocyclic ring.

In a suitable embodiment of the invention X can be O, S, NH, or NCN. In more suitable embodiments X is O or S.

Specific embodiments of the invention include, but are not limited to, the following compounds of formula 1:

2-[3-(2-methoxy-5-nitro-phenyl)-thioureido]-N-(2-indanyl)-2-(3-thienyl) acetamide E42.2

2-[3-(2-methoxy-5-nitro-phenyl)-thioureido]-N-(3,4-dimethylphenyl)-2-phenyl acetamide E32.2

2-[3-(2-methoxy-5-nitro-phenyl)-ureido]-N-(3,4-dimethylphenyl)-2-phenyl acetamide E32.5

(R)-2-[3-(2-methoxy-5-nitro-phenyl)-thioureido]-N-(3,4-dimethylphenyl)-2-phenyl acetamide E33.1

2-[3-(2-methoxy-5-nitro-phenyl)-ureido]-N-(2-indanyl)-2-(3-thienyl) acetamide E42.1

(R)-2-[3-(2-nitro-5-methoxy-phenyl)-ureido]-N-(2-indanyl)-2-phenyl acetamide E29.1

(R)-2-[3-(2-nitro-5-methoxy-phenyl)-ureido]-N-(4-chlorophenyl)-2-phenyl acetamide E4.1

and (R)-2-[3-(2-methoxy-5-nitro-phenyl)-ureido]-N-(3-trifluromethylphenyl)-2-phenyl acetamide E31.2

In another embodiment of the invention, the compound of Formula I is provided in labeled form, such as radiolabeled form (e.g. labeled by incorporation within its structure ³H or ¹⁴C or by conjugation to ¹²⁵I). In a preferred aspect of the invention, such compounds, which bind preferentially to GlyT-1, can be used to identify GlyT-1 receptor ligands by techniques common in the art. This can be achieved by incubating the receptor or tissue in the presence of a ligand candidate and then incubating the resulting preparation with an equimolar amount of radiolabeled compound of the invention. GlyT-1 receptor ligands are thus revealed as those that significantly occupy the GlyT-1 site and prevent binding of the radiolabeled compound of the present invention. Alternatively, GlyT-1 receptor ligand candidates may be identified by first incubating a radiolabeled form of a compound of the invention then incubating the resulting preparation in the presence of the candidate ligand. A more potent GlyT-1 receptor ligand will, at equimolar concentration, displace the radiolabeled compound of the invention.

Acid addition salts of the compounds of Formula I are most suitably formed from pharmaceutically acceptable acids, and include for example those formed with inorganic acids e.g. hydrochloric, sulphuric or phosphoric acids and organic acids e.g. succinic, maleic, acetic or fumaric acid. Other non-pharmaceutically acceptable salts e.g. oxalates may be used for example in the isolation of compounds of Formula I for laboratory use, or for subsequent conversion to a pharmaceutically acceptable acid addition salt. Also included within the scope of the invention are base addition salts such as sodium, potassium and ammonium salts, solvates and hydrates of compounds of the invention.

The conversion of a given compound to a desired compound salt is achieved by applying standard techniques, well known to one skilled in the art.

Compounds which inhibit GlyT-1 mediated glycine transport will increase glycine concentrations at NMDA receptors, which receptors are located in the forebrain, among other locations. This concentration increase elevates the activity of NMDA receptors, thereby alleviating schizophrenia and enhancing cognitive function. Alternatively, compounds that interact directly with the glycine receptor component of the NMDA receptor can have the same or similar effects as increasing or decreasing the availability of extracellular glycine caused by inhibiting or enhancing GlyT-1 activity, respectively. See, for example, Pitkänen et al., Eur. J. Pharmacol., 253, 125-129 (1994); Thiels et al., Neuroscience, 46, 501-509 (1992); and Kretschmer and Schmidt, J. Neurosci., 16, 1561-1569 (1996).

For use in medicine, the compounds of the present invention can be administered in a standard pharmaceutical composition. The present invention therefore provides, in a further aspect, pharmaceutical compositions comprising a pharmaceutically acceptable carrier and a Formula 3 compound or a pharmaceutically acceptable salt, solvate or hydrate thereof, in an amount effective to treat the target indication.

The compounds of the invention are, for instance, administered orally, sublingually, rectally, nasally, vaginally, topically (including the use of a patch or other transdermal delivery device), by pulmonary route by use of an aerosol, or parenterally, including, for example, intramuscularly, subcutaneously, intraperitoneally, intraarterially, intravenously or intrathecally. Administration can be by means of a pump for periodic or continuous delivery. The compounds of the invention are administered alone, or are combined with a pharmaceutically-acceptable carrier or excipient according to standard pharmaceutical practice. For the oral mode of administration, the compounds of the invention are used in the form of tablets, capsules, lozenges, chewing gum, troches, powders, syrups, elixirs, aqueous solutions and suspensions, and the like. In the case of tablets, carriers that are used include lactose, sodium citrate and salts of phosphoric acid. Various disintegrants such as starch, and lubricating agents such as magnesium stearate and talc, are commonly used in tablets. For oral administration in capsule form, useful diluents are lactose and high molecular weight polyethylene glycols. If desired, certain sweetening and/or flavoring agents are added. For parenteral administration, sterile solutions of the compounds of the invention are usually prepared, and the pHs of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzylchromium chloride, and the usual quantities of diluents and/or carriers. For pulmonary administration, diluents and/or carriers will be selected to be appropriate to allow the formation of an aerosol.

Suppository forms of the compounds of the invention are useful for vaginal, urethral and rectal administrations. Such suppositories will generally be constructed of a mixture of substances that is solid at room temperature but melts at body temperature. The substances commonly used to create such vehicles include theobroma oil, glycerinated gelatin, hydrogenated vegetable oils, mixtures of polyethylene glycols of various molecular weight and fatty acid esters of polyethylene glycol. See, Remington's Pharmaceutical Sciences, 16th Ed., Mack Publishing, Easton, Pa., 1980, pp. 1530-1533 for further discussion of suppository dosage forms and other dosage forms. Analogous gels or creams can be used for vaginal, urethral and rectal administrations.

Numerous administration vehicles will be apparent to those of ordinary skill in the art, including without limitation slow release formulations, liposomal formulations and polymeric matrices.

Examples of pharmaceutically acceptable acid addition salts for use in the present invention include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic and arylsulphonic acids, for example. Examples of pharmaceutically acceptable base addition salts for use in the present invention include those derived from non-toxic metals such as sodium or potassium, ammonium salts and organoamino salts such as triethylamine salts. Numerous appropriate such salts will be known to those of ordinary skill.

The physician or other health care professional can select the appropriate dose and treatment regimen based on the subject's weight, age, and physical condition. Dosages will generally be selected to maintain a serum level of compounds of the invention between about 0.01 μg/cc and about 1000 μg/cc, preferably between about 0.1 μg/cc and about 100 μg/cc. For parenteral administration, an alternative measure of preferred amount is from about 0.001 mg/kg to about 10 mg/kg (alternatively, from about 0.01 mg/kg to about 10 mg/kg), more preferably from about 0.01 mg/kg to about 1 mg/kg (from about 0.1 mg/kg to about 1 mg/kg), will be administered. For oral administrations, an alternative measure of preferred administration amount is from about 0.001 mg/kg to about 10 mg/kg (from about 0.1 mg/kg to about 10 mg/kg), more preferably from about 0.01 mg/kg to about 1 mg/kg (from about 0.1 mg/kg to about 1 mg/kg). For administrations in suppository form, an alternative measure of preferred administration amount is from about 0.1 mg/kg to about 10 mg/kg, more preferably from about 0.1 mg/kg to about 1 mg/kg.

For use in assaying for activity in inhibiting glycine transport, eukaryokic cells, preferably QT-6 cells derived from quail fibroblasts, have been transfected to express one of the three known variants of human GlyT-1, namely GlyT-1a, GlyT-1b or GlyT-1c, or human GlyT-2. The sequences of these GlyT-1 transporters are described in Kim et al., Molec. Pharm. 45: 608-617, 1994, excepting that the sequence encoding the extreme N-terminal of GlyT-1a was merely inferred from the corresponding rat-derived sequence. This N-terminal protein-encoding sequence has now been confirmed to correspond to that inferred by Kim et al. The sequence of the human GlyT-2 is described by Albert et al., U.S. Pat. No. 919,653 issued July 1999, which is incorporated herein by reference in its entirety. Suitable expression vectors include pRc/CMV (Invitrogen), Zap Express Vector (Stratagene Cloning Systems, LaJolla, Calif.; hereinafter “Stratagene”), pBk/CMV or pBk-RSV vectors (Stratagene), Bluescript II SK +/− Phagemid Vectors (Stratagene), LacSwitch (Stratagene), pMAM and pMAM neo (Clontech), among others. A suitable expression vector is capable of fostering expression of the included GlyT DNA in a suitable host cell, preferably a non-mammalian host cell, which can be eukaryotic, fungal, or prokaryotic. Such preferred host cells include amphibian, avian, fungal, insect, and reptilian cells.

Compounds of the present invention (compounds of Formula 1) can be made by the method shown in Scheme I starting from the appropriate amino acid A. The amino acid A is Boc protected at the Nitrogen atom to give the intermediate B. A method for carrying out the Boc protection is shown as I in Scheme 1. The Boc protected amino acid can then be reacted with a primary amine as in method 11 to give the amide intermediate C. The Boc group is then removed to give the free amine D, which can then be reacted an with an isocyanate or isothiocyanate to give the urea or thiourea product E respectively.

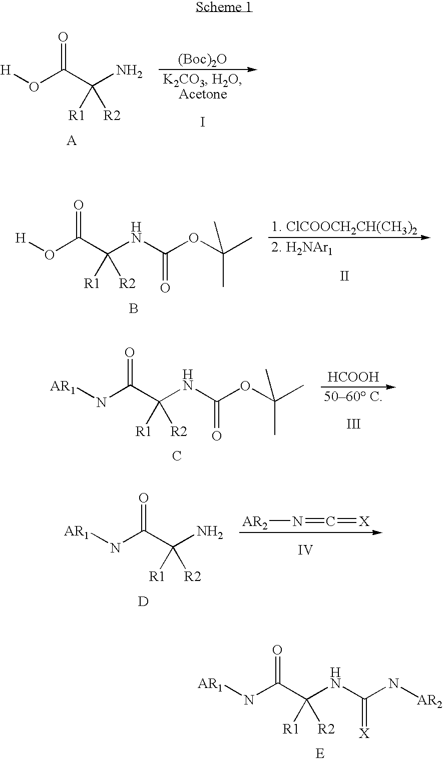

### EXAMPLES

General Procedures

I- Conversion of Amino acid A to Boc protected product B

To a round bottom flask was added the amino acid (1 eq.), Et₃N (5 eq.) 1 M NaOH (1 eq.) and CH₃CN. The clear solution was cooled down to 0° C. and to it was added (Boc)₂O. The reaction was warmed to room temperature and stirred for four hours, during which time a white precipitate formed. The reaction mixture was concentrated and the residue was dissolved in EtOAc:water (1:1). The organic phase was washed with water and the aqueous phases were combined and treated with 10% HCl and then were extracted with EtOAc three times. The combined organic phase was washed successively with water, brine, dried over MgSO₄, filtered and concentrated to yield the title compound.

II- Formation of amide intermediate C from Boc protected Amino Acid, B, and a primary Amine.

To a flame dried round bottom flask was added Boc protected-amino acid and CH₂Cl₂ (5 mL). The clear solution was cooled to 0° C. and the primary amine (1 eq.) was added followed by diisopropylethylamine (2 eq.) and N,N-bis(2-oxo-3-oxazolidinyl)phosphonic chloride (1 eq.). The white suspension was allowed to stir at 0° C. under Argon for two hours. The workup included pouring the clear reaction mixture into ether (3 parts) and water (2 parts). The organic layer was separated and was successively washed with 1 N NaHSO₄, water, sat. NaHCO₃ and brine. It was dried over MgSO₄, filtered and concentrated to yield the title compound.

III- Boc-deprotection of amide C to give intermediate D

The intermediate amide C and formic acid (neat) were added to a sealed vial. The vial was heated at 60° C. in an oil bath for forty minutes. After cooling, the reaction mixture was concentrated and the residue was purified by an SPE tube using first CH₂Cl₂:MeOH 98:2 followed by 95:5, 94:6, 93:7, 92:8, 90:10, 85:15, 80:20 and finally 100% MeOH to yield the title compound.

IV- Formation of final product E from D and an isocyanate or isothiocyanate.

The amine D (1 eq.), and the desired isocyanate or isothiocyanate (1.2 eq.), triethylamine (1 drop) and acetone (2 mL) were added to a sealed vial. The reaction was heated to 50° C. and was left stirring for four hours. The mixture was concentrated and the crude product was purified by an SPE tube using Hexanes:Ethyl Acetate (90:10), (80:20), (70:30), (60:40), (50:50) and finally (40:60) as the eluent to yield the title compound.

The compounds of examples 1 through 41 were made from the indicated starting materials by the general synthetic procedures described above unless otherwise noted.

Experimental

In the experimental section each experiment describes the formation of a series of intermediate compounds (A, B, C, and D) and the formation of one or more final products, E , by the reaction of intermediate D with one or more reagents. When more than one final product E is made from a particular intermediate D the final products are given differentiating numbers after the decimal point. (for example E1.1 and E1.2). While the general scheme shows four steps for making the final product some of the required intermediates were found to be commercially available. In cases where an intermediate is commercially available the experiment starts with that intermediate (not with A). For example Experiment 1 begins with the commercially available intermediate C1 which is converted to D1 which is then reacted with two different reagents to produce two different final products E1.1 and E1.2. Finally some of the compounds were produced as single enantiomers while others were produced as a mixture. The numbering system indicates the (R) enantiomer with a * and the (S) enantiomer with **.

Experiment 1:

B1 N-Tert-butoxycarbony DL-phenyl glycine

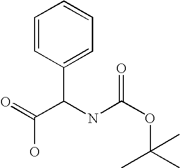

N-Tert-butoxycarbonyl DL-phenyl glycine was isolated as a white solid (7.61 g, 92%) from DL-2-phenylglycine (5.0 g, 33.1 mmol), 1 N NaOH (132.4 mL, 132.4 mmol) and (BOC)₂O (19.0 mL, 82.7 mmol).

C1 tert-butyl [1-(2-methylphenylcarbamoyl)-1-phenyl-methyl]-carbamate

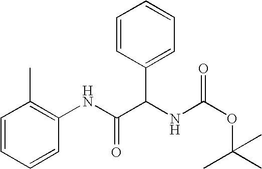

[(tert-butoxycarbonyl)amino](phenyl)acetic acid (2.50 g, 9.95 mmol) was dissolved in dry THF (27 mL) in a flame dried flask under Argon. The solution was cooled to −50° C. and N-methylmorpholine (1.11 g, 10.95 mmol) and isobutylchloroformate (1.50 g, 10.95 mmol) were added. The reaction was allowed to stir at this temperature for 2.5 hours. N-methylmorpholine (1.20 g, 11.94 mmol) was added to o-toluidine in THF (3 mL). This solution was added to the reaction and the reaction was stirred overnight at which time it warmed to room temperature. The THF was then evaporated and CH₂Cl₂ (250 mL) was added. The solution was poured into a separatory funnel and NaHCO₃ (sat.) was added. The organic phase was isolated and washed with NaHCO₃ (sat), water and brine. The organic layer was then dried over Na₂SO₄, filtered and concentrated to yield a white solid (3.32 g, 98%).

D1 2-Amino-N-(2-methylphenyl)-2-phenylacetamide

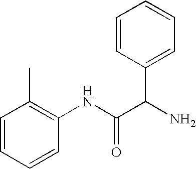

tert-butyl [1-(2-methylphenylcarbamoyl)-1-phenyl-methyl]-carbamate (2.00 g, 5.88 mmol) was dissolved in formic acid (20 mL) and the solution was allowed to stir for 2 hours at 50° C. under Argon. The flask was cooled to room temperature and the formic acid was evaporated. The resultant oil was dissolved in CH₂Cl₂ and poured into a separatory funnel. 1 N NaOH was added and the product was extracted with CH₂Cl₂ three times. The combined organic layers were washed with water and brine, dried over NaSO₄, filtered and concentrated. The resultant oil was dissolved in a small amount of EtOAc and Hexane was added slowly. The solution became cloudy and a white solid precipitated (780.0 mg, 56%). The mother liquor was removed by pipette and the solid was washed with Hexane three times and was dried under vacuum.

E1.1 N-(2-methylphenyl)-2-[3-(2-methoxy-5-nitrophenyl)-thioureido]-2-phenyl acetamide

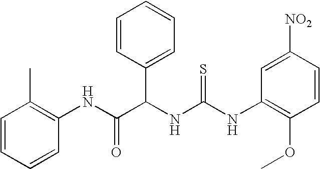

N-(2-methylphenyl)-2-[3-(2-methoxy-5-nitrophenyl)-thioureido]-2-phenyl acetamide (18.3 mg, 38%) was isolated as an off white solid from 2-Amino-N-(2-methylphenyl)-2-phenylacetamide (25.6 mg, 0.107 mmol) and 2-methoxy-5-nitrophenyl isothiocyanate (33.8 mg, 0.149 mmol).

E1.2 N-(2-methylphenyl)-2-[3-(3-fluorophenyl)-thioureido]-2-phenyl acetamide

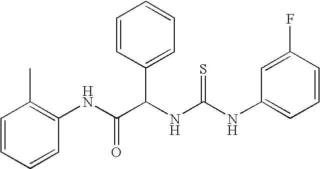

N-(2-methylphenyl)-2-[3-(3-fluorophenyl)-thioureido]-2-phenyl acetamide (5.9 mg, 14%) was isolated as a white solid from 2-Amino-N-(2-methylphenyl)-2-phenylacetamide (25.7 mg, 0.107 mmol) and 3-fluorophenyl isothiocyanate (24.7 mg, 0.161 mmol).

E1.3N-(2-methylphenyl)-2-[3-(2-methoxyphenyl)-thioureido]-2-phenyl acetamide

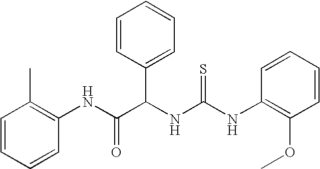

N-(2-methylphenyl)-2-[3-(2-methoxyphenyl)-thioureido]-2-phenyl acetamide (18.1 mg, 38%) was isolated as a white solid from 2-Amino-N-(2-methylphenyl)-2-phenylacetamide (28.4 mg, 0.118 mmol) and 2-methoxyphenyl isothiocyanate (31.7 mg, 0.177 mmol).

E1.4 N-(2-methylphenyl)-2-[3-(4-fluorophenyl)-thioureido]-2-phenyl acetamide

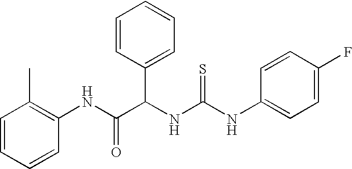

N-(2-methylphenyl)-2-[3-(4-fluorophenyl)-thioureido]-2-phenyl acetamide (19.9 mg, 46%) was isolated as a white solid from 2-Amino-N-(2-methylphenyl)-2-phenylacetamide (26.1 mg, 0.109 mmol) and 4-fluorophenyl isothiocyanate (25.1 mg, 0.164 mmol).

E1.5 N-(2-methylphenyl)-2-[3-(3-methoxyphenyl)-thioureido]-2-phenyl acetamide

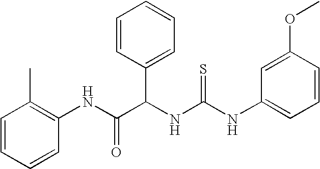

N-(2-methylphenyl)-2-[3-(3-methoxyphenyl)-thioureido]-2-phenyl acetamide (40.6 mg, 76%) was isolated as a white solid from 2-Amino-N-(2-methylphenyl)-2-phenylacetamide (31.4 mg, 0.131 mmol) and 3-methoxyphenyl thioisocyanate (35.3 mg, 0.197 mmol).

E1.6 N-(2-methylphenyl)-2-[3-(4-methoxyphenyl)-thioureido]-2-phenyl acetamide

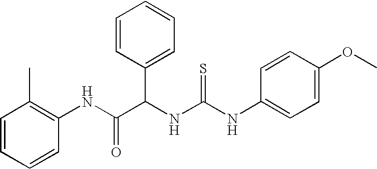

N-(2-methylphenyl)-2-[3-(4-methoxyphenyl)-thioureido]-2-phenyl acetamide (10.8 mg, 19%) was isolated as a white solid from 2-Amino-N-(2-methylphenyl)-2-phenylacetamide (33.4 mg, 0.139 mmol) and 4-methoxyphenyl thioisocyanate (37.5 mg, 0.209 mmol).

E1.7 N-(2-methylphenyl)-2-[3-phenylthioureido]-2-phenyl acetamide

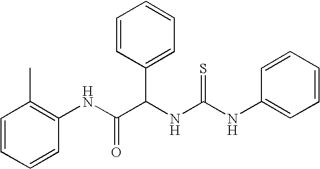

N-(2-methylphenyl)-2-[3-phenylthioureido]-2-phenyl acetamide was isolated as a white solid, (20.6 mg, 69%) from phenylisothiocyanate (17 mg, 0.13 mmol) and N-(2-methylphenyl)phenylglycinamide (20 mg, 0.08 mmol).

E1.8 N-(2-methylphenyl)-2-[3-(3-trifluoromethylphenyl)-thioureido]-2-phenyl acetamide

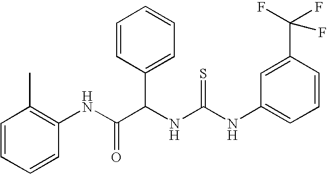

N-(2-methylphenyl)-2-[3-(3-trifluoromethylphenyl)-thioureido]-2-phenyl acetamide was isolated as a white solid, (30 mg, 85%) from 3-trifluorophenylisothiocyanate (24 mg, 0.13 mmol) and N-(2-methylphenyl)phenylglycinamide (20 mg, 0.08 mmol).

Experiment 2:

B2 1-(Tert-butoxycarbonylamino) cyclohexanecarboxylic acid

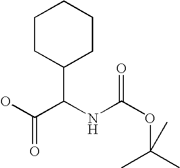

Tetramethylammonium hydroxide (1.27 g, 6.98 mmol) was added to 1-amino-1-cyclohexane carboxylic acid (1.0 g, 6.98 mmol) in CH₃CN (20 mL). The mixture was allowed to stir for 45 minutes at which time (Boc)₂O (3.05 g, 13.97 mmol) was added and the reaction was allowed to stir at room temperature for three hours. The solvent was then evaporated and Et₂O was added. The Et₂O layer was extracted with water twice. The combined water layers were then acidified with 10% HCl and EtOAc was added. The product was extracted with EtOAc three times. The combined EtOAc layers were washed with brine, dried over Na₂SO₄, filtered and concentrated to yield the title compound (630.0 mg, 37%) as a white solid.

C2 Tert butyl-[1-(2-cyclohexyl-carbamoyl)-1-phenylmethyl] carbamate

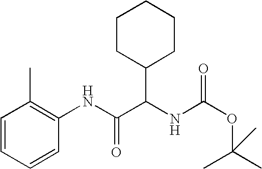

To a round bottom flask was added 1-(Tert-butoxycarbonylamino) cyclohexanecarboxylic acid (630.0 mg, 2.59 mmol), o-toluidine (0.31 g, 2.85 mmol) and 1 -methylimidazole (0.43 g, 5.19 mmol) in DMF (15 mL) under Argon. Diethylcyanophosphonate was added dropwise while the flask was cooled in ice. The reaction was allowed to stir for three days during which time it warmed to room temperature. NaHCO₃ (sat) was added and a white precipitate formed. The reaction was poured into a separatory funnel and 1 M NaHSO₄ was added and the precipitate dissolved. The product was extracted with EtOAc three times and the combined organic layers were washed with brine, dried over NaSO₄, filtered and concentrated. The crude product was purified by column chromatography (15% EtOAc in Hexanes) to yield the title compound (230 mg, 27%) as a white solid.

D2 1-Amino-N-(2-methylphenyl)cyclohexane carboxamide

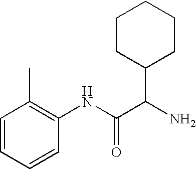

1-Amino-N-(2-methylphenyl)cyclohexane carboxamide (564.0 mg, 79%) was isolated as a white solid from 1 -tert butyl-N-(2-methylphenyl)cyclohexane carbamate (0.850 g, 2.56 mmol).

E2.1 [1-(3-fluorophenyl)-thioureido N-(2-methylphenyl)]-cyclohexane carboxamide

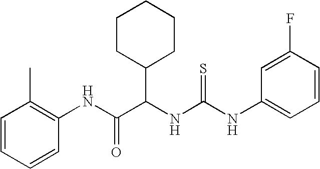

1-Amino-N-(2-methylphenyl)cyclohexane carboxamide (22.1 mg, 0.144 mmol) was dissolved in CH₂Cl₂ (1.0 mL) and Et₃N (1.0 mL) was added followed by 3-fluorophenylisothiocyanate (22.1 mg, 0.144 mmol) . The reaction was allowed to stir at 40° C. for three hours. EtOAc was added (˜20 mL) and the reaction was poured into a separatory funnel. 1 M HCl was added and the aqueous layer was extracted with EtOAc three times. The combined organic layers were washed with brine, dried over NaSO₄, filtered and concentrated. The crude product was purified by an SPE tube using 50% EtOAc in hexanes as the eluent to yield the title compound (4.2 mg, 15%) as a yellow film.

E2.2 [1-(4-nitrophenyl)-thioureido]-N-(2-methylphenyl)-cyclohexane carboxamide

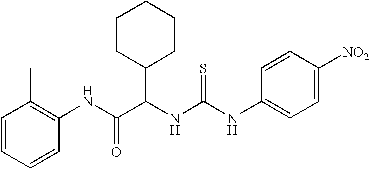

1-Amino-N-(2-methylphenyl)cyclohexane carboxamide (20.4 mg, 0.073 mmol) was dissolved in CH₂Cl₂ (2.0 mL) and Et₃N (8.9 mg, 0.088 mmol) was added followed by 4-nitrophenyl isothiocyanate (15.9 mg, 0.088 mmol). The reaction was allowed to stir in a sealed tube for three hours. The reaction was then concentrated and purified by an SPE tube using first 30% EtOAc/Hexanes followed by 50% EtOAc/Hexanes followed by 100% EtOAc. The title compound was isolated as a yellow solid (6.6 mg, 22%).

E2.3 [1-(3-nitrophenyl)-thioureido]-N-(2-methylphenyl)-cyclohexane carboxamide

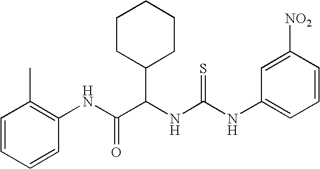

[1-(3-nitrophenyl)-thioureido]-N-(2-methylphenyl)-cyclohexane carboxamide (21.5 mg, 74%) was isolated as a yellow solid from 1-Amino-N-(2-methylphenyl)cyclohexane carboxamide (19.4 mg, 0.070 mmol) and 3-nitrophenyl isothiocyanate (15.1 mg, 0.084 mmol).

E2.4 [1-(4-methoxyphenyl)-thioureido]-N-(2-methylphenyl)-cyclohexane carboxamide

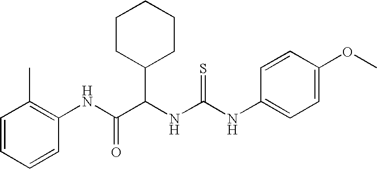

[1-(4-methoxyphenyl)-thioureido]-N-(2-methylphenyl)-cyclohexane carboxamide (14.0 mg, 32%) was isolated as an off white solid from 1-Amino-N-(2-methylphenyl)cyclohexane carboxamide (30.5 mg, 0.110 mmol) and 4-methoxyphenylisothiocyanate (21.8 mg, 0.132 mmol).

E2.5 [1-(2-fluorophenyl)-thioureido-N-(2-methylphenyl)]cyclohexane carboxamide

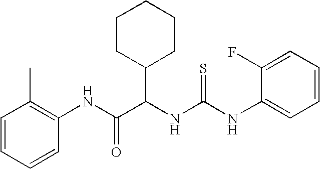

[1-(2-fluorophenyl)-thioureido-N-(2-methylphenyl)]cyclohexane carboxamide (5.6 mg, 16%) was isolated as brown solid from 1-Amino-N-(2-methylphenyl)cyclohexane carboxamide (25.4 mg, 0.091 mmol) and 2-fluorophenyl isothiocyanate (27.8 mg, 0.182 mmol).

E2.6 [1-(4-fluorophenyl)-thioureido]-N-(2-methylphenyl)-cyclohexane carboxamide

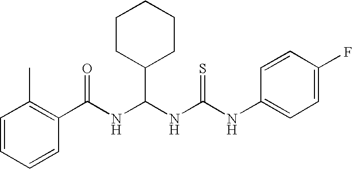

[1-(4-fluorophenyl)-thioureido]-N-(2-methylphenyl)-cyclohexane carboxamide (2.9 mg, 10%) from 1-Amino-N-(2-methylphenyl)cyclohexane carboxamide (20.6 mg, 0.074 mmol) and 4-fluorophenyl isothiocyanate (22.7 mg, 0.148 mmol).

Experiment 3:

C3 tert-butyl [1-(3-chlorophenylcarbamoyl)-1-phenyl-methyl]-carbamate

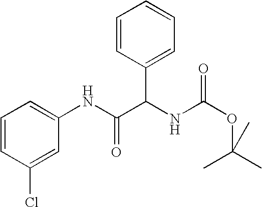

tert-butyl [1-(3-chlorophenylcarbamoyl)-1-phenyl-methyl]-carbamate was isolated as a white solid (289.5 mg, 81%) from N- Tert-butoxycarbonyl DL-phenyl glycine (250.0 mg, 0.99 mmol), N-methylmorpholine (0.12 mL, 1.09 mmol), isobutylchloroformate (0.14 mL, 1.09 mmol), 3-chloroaniline (0.13 mL, 1.19 mmol) and N-methylmorpholine (0.13 mL, 1.19 mmol).

D3 2-Amino-N-(3-chlorophenyl)-2-phenylacetamide

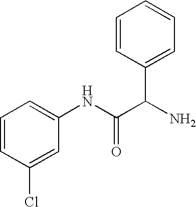

2-Amino-N-(3-chlorophenyl)-2-phenylacetamide was isolated as a white solid (138.6 mg, 62%) from tert-butyl [1-(4-chlorophenylcarbamoyl)-1-phenyl-methyl]-carbamate (280.2 mg, 0.86 mmol).

E3.1 N-(3-chlorophenyl)-2-[3-(2-methoxy-5-nitrophenyl)-thioureido]-2-phenyl acetamide

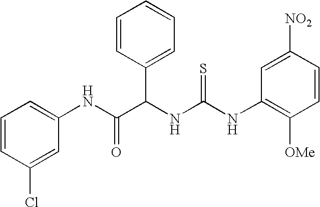

N-(3-chlorophenyl)-2-[3-(2-methoxy-5-nitrophenyl)-thioureido]-2-phenyl 10 acetamide was isolated as a light yellow solid (21.9 mg, 58%) from 2-Amino-N-(3-chlorophenyl)-2-phenylacetamide (20.0 mg, 0.08 mmol) and 2-methoxy-5-nitrophenylisothiocyanate (21.0 mg, 0.10 mmol).

Experiment 4:

C4 tert-butyl [1-(4-chlorophenylcarbamoyl)-1-phenyl-methyl]-carbamate

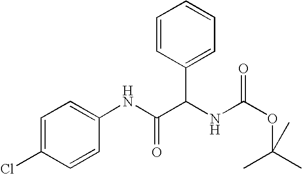

tert-butyl [1-(4-chlorophenylcarbamoyl)-1-phenyl-methyl]-carbamate was isolated as a white solid (266.2 mg, 74%) from N-Tert-butoxycarbonyl DL-phenyl glycine (250.0 mg, 0.99 mmol), N-methylmorpholine (0.12 mL, 1.09 mmol), isobutylchloroformate (0.14 mL, 1.09 mmol), 4-chloroaniline 9152.3 mg, 1.19 mmol) and N-methylmorpholine (0.13 mL, 1.19 mmol).

D4 2-Amino-N-(4-chlorophenyl)-2-phenylacetamide

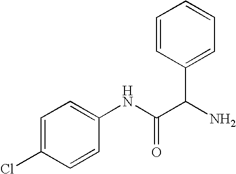

2-Amino-N-(4-chlorophenyl)-2-phenylacetamide was isolated as a white solid (150.3 mg, 72%) from tert-butyl [1-(4-chlorophenylcarbamoyl)-1-phenyl-methyl]-carbamate (259.6 mg, 0.80 mmol).

E4.1 N-(4-chlorophenyl)-2-[3-(2-methoxy-5-nitrophenyl)-thioureido]-2-phenyl acetamide

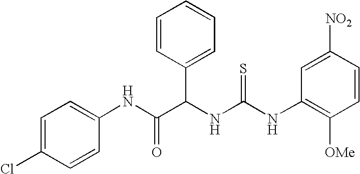

N-(4-chlorophenyl)-2-[3-(2-methoxy-5-nitrophenyl)-thioureido]-2-phenyl acetamide was isolated as a light yellow solid (20.0 mg, 53%) from 2-Amino-N-(4-chlorophenyl)-2-phenylacetamide (20.0 mg, 0.08 mmol) and 2-methoxy-5-nitrophenylisothiocyanate (21.0 mg, 0.10 mmol).

E4.2 2-[3-(2-methoxy-5-nitro-phenyl)-ureido]-N-(4-chlorophenyl)-2-phenyl acetamide

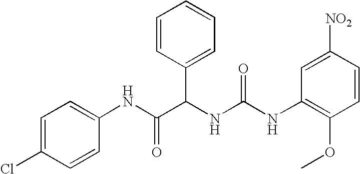

2-[3-(2-methoxy-5-nitro-phenyl)-ureido]-N-(4-chlorolphenyl)-2-phenyl acetamide (25.2 mg, 95.5%); from 2-Amino-N-(4-chlorophenyl)-2-phenylacetamide (15 mg, 0.058 mmoles) reacted with 2-methoxy-5-nitrophenylisocyanate (15 mg, 0.077 mmoles) in dichloromethane (0.8 ml) at 60° C. overnight.

C4* R-tert-butyl [1-(4-chlorophenylcarbamoyl)-1-phenyl-methyl]-carbamate

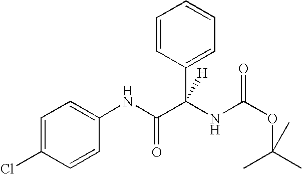

R-tert-butyl [1-(4-chlorophenylcarbamoyl)-1-phenyl-methyl]-carbamate was isolated as a white solid (342.2 mg) from N-Tert-butoxycarbonyl D-phenyl glycine (600.0 mg, 2.39 mmol), N-methylmorpholine (0.288 mL, 2.62 mmol), isobutylchloroformate (0.339 mL, 2.62 mmol), 4-chloroaniline (418 mg, 3.144 mmol) and N-methylmorpholine (0.318 mL, 3.144 mmol) and THF (2 ml).

D4* R-2-Amino-N-(4-chlorophenyl)-2-phenylacetamide

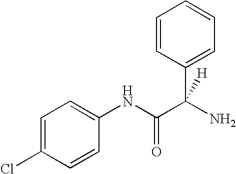

R-2-Amino-N-(4-chlorophenyl)-2-phenylacetamide was isolated as a white solid (484.6 mg) from R-tert-butyl [1-(4-chlorophenylcarbamoyl)-1-phenyl-methyl]-carbamate (342.2 mg) and Formic acid (2 ml).

E4.1* R-2-[3-(2-methoxy-5-nitro-phenyl)-ureido]-N-(4-chlorophenyl)-2-phenyl acetamide

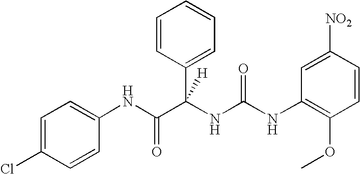

R-2-[3-(2-methoxy-5-nitro-phenyl)-ureido]-N-(4-chlorolphenyl)-2-phenyl acetamide (280.0 mg, 53.4%); from R-2-Amino-N-(4-chlorophenyl)-2-phenylacetamide (300 mg, 1.151 mmoles) reacted with 2-methoxy-5-nitrophenylisocyanate (290 mg, 1.495 mmoles) in dichloromethane (10 ml) at 60° C. overnight.

Experiment 5:

C5 tert-butyl [1-(2.3-dimethylphenylcarbamoyl)-1-phenyl-methyl]-carbamate

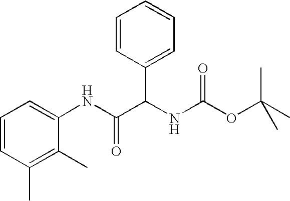

tert-butyl [1-(2,3-dimethylphenylcarbamoyl)-1-phenyl-methyl]-carbamate was isolated as a white solid (230.5 mg, 66%) from BOC-phenylglycine (250.0 mg, 0.99 mmol), N-methylmorpholine (0.12 mL, 1.09 mmol), isobutylchloroformate (0.14 mL, 1.09 mmol), 2,3-dimethylaniline (0.15 mL, 1.19 mmol) and N-methylmorpholine (0.13 mL, 1.19 mmol).

D5 2-Amino-N-(2,3-dimethylphenyl)-2-phenylacetamide

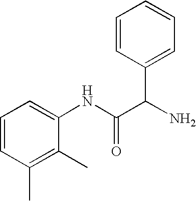

2-Amino-N-(2,3-dimethylphenyl)-2-phenylacetamide was isolated as a white solid (118.6 mg, 75%) from tert-butyl [1-(2,3-dimethylphenylcarbamoyl)-1-phenyl-methyl]-carbamate (220.4 mg, 0.62 mmol).

E5.1 N-(2,3-dimethylphenyl)-2-[3-(2-methoxy-5-nitrophenyl)-thioureido]-2-phenyl acetamide

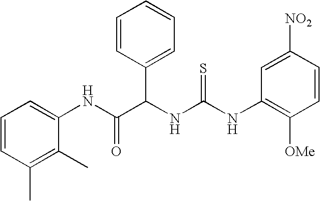

N-(2,3-dimethylphenyl)-2-[3-(2-methoxy-5-nitrophenyl)-thioureido]-2-phenyl acetamide was isolated as a light yellow solid (16.7 mg, 60%) from 2-Amino-N-(2,3-dimethylphenyl)-2-phenylacetamide (14.6 mg, 0.06 mmol) and 2-methoxy-5-nitrophenylisothiocyanate (915.7 mg, 0.07 mmol).

Experiment 6:

C6 tert-butyl [1-(5,6,7,8-tetrahydro-1-naphthylcarbamoyl)-1-phenyl-methyl]-carbamate

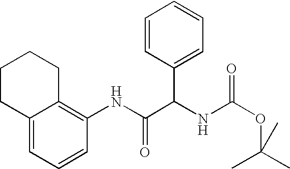

tert-butyl [1-(5,6,7,8-tetrahydro-1-naphthylcarbamoyl)-1-phenyl-methyl]-carbamate was isolated as a white solid (299.8 mg, 80%) from BOC-phenyl glycine (250.0 mg, 0.99 mmol), N-methylmorpholine (0.12 mL, 1.09 mmol), isobutylchloroformate (0.14 mL, 1.09 mmol), 5,6,7,8-tetrahydro-1-naphthlamine (175.8 mg, 1.19 mmol) and n-methylmorpholine (0.13 mL, 1.19 mmol).

D6 2-Amino-N-(5,6.7,8-tetrahydronaphthyl)-2-phenylacetamide

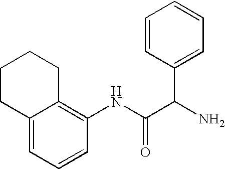

2-Amino-N-(5,6,7,8-tetrahydronaphthyl)-2-phenylacetamide was isolated as a white solid (147.9 mg, 70%) from tert-butyl [1-(5,6,7,8-tetrahydro-1-naphthylcarbamoyl)-1-phenyl-methyl]-carbamate (286.9 mg, 0.75 mmol).

E6.1 N-(5,6,7,8,-tetrahydronaphthyl)-2-[3-(2-methoxy-5-nitrophenyl)-thioureido]-2-phenyl acetamide

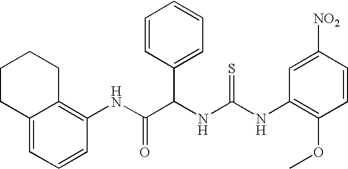

N-(5,6,7,8,-tetrahydronaphthyl)-2-[3-(2-methoxy-5-nitrophenyl)-thioureido]-2-phenyl acetamide was isolated as a light yellow solid (19.2 mg, 65%) from 2-Amino-N-(5,6,7,8,-tetrahydronaphthyl)-2-phenylacetamide (16.1 mg, 0.06 mmol) and 2-methoxy-5-nitrophenylisothiocyanate (15.7 mg, 0.07 mmol).

Experiment 7:

C7 tert-butyl [1-(2-methyl-4-chlorophenylcarbamoyl)-1-phenyl-methyl]-carbamate

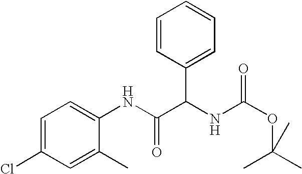

tert-butyl [1-(2-methyl-4-chlorophenylcarbamoyl)-1-phenyl-methyl]-carbamate was isolated as white solid (296.7 mg, 80%) from BOC-phenylglycine (250.0 mg, 0.99 mmol), N-methylmorpholine (0.12 mL, 1.09 mmol), isobutylchloroformate (0.14 mL, 1.09 mmol), -2-methyl-4-chloroaniline (169.1 mg, 1.19 mmol) and N-methylmorpholine (0.13 mL, 1.19 mmol).

D7 2-Amino-N-(2-methyl-4-chlorophenyl)-2-phenylacetamide

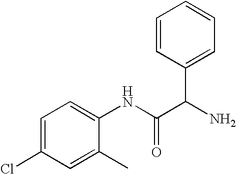

2-Amino-N-(2-methyl-4-chlorophenyl)-2-phenylacetamide was isolated as a white solid (148.3 mg, 71%) from tert-butyl [1-(2-methyl-4-chlorophenylcarbamoyl)-1-phenyl-methyl]-carbamate (284.6 mg, 0.76 mmol).

E7.1 N-(2-methyl-4-chlorophenyl)-2-[3-(2-methoxy-5-nitrophenyl)-thioureido]-2-phenyl acetamide

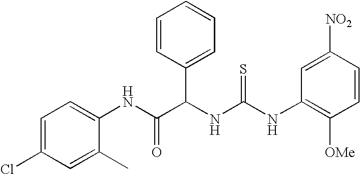

N-(2-methyl-4-chlorophenyl)-2-[3-(2-methoxy-5-nitrophenyl)-thioureido]-2-phenyl acetamide was isolated as a light yellow solid (19.0 mg, 65%) from 2-Amino-N-(2-methyl-4-chlorophenyl)-2-phenylacetamide (15.8 mg, 0.06 mmol) and 2-methoxy-5-nitrophenylisothiocyanate (15.7 mg, 0.07 mmol).

Experiment 8:

C8 tert-butyl [1-(2-ethyl-6-methylphenylcarbamoyl)-1-phenyl-methyl]-carbamate

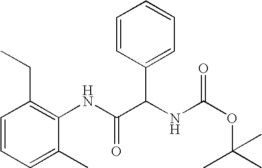

tert-butyl [1-(2-ethyl-6-methylphenylcarbamoyl)-1-phenyl-methyl]-carbamate was isolated as a white solid (236.6 mg, 65%) from BOC-phenylglycine (250.0 mg, 0.99 mmol), N-methylmorpholine (0.12 mL, 1.09), isobutylchloroformate (0.14 mL, 1.09 mmol), 6-ethyl-o-toluidine (0.17 mL, 1.19 mmol) and N-methylmorpholine (0.13 mL, 1.19 mmol).

D8 2-Amino-N-(2-ethyl-6-methylphenyl)-2-phenylacetamide

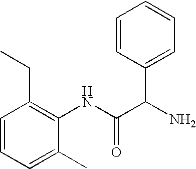

2-Amino-N-(2-ethyl-6-methylphenyl)-2-phenylacetamide was isolated as a white solid (83.5 mg, 50%) from tert-butyl [1-(2-ethyl-6-methylphenylcarbamoyl)-1-phenyl-methyl]-carbamate (228.2 mg, 0.62 mmol).

E8.1 N-(2-ethyl-6-methylphenyl)-2-[3-(2-methoxy-5-nitrophenyl)-thioureido]-2-phenylacetamide

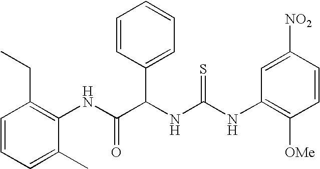

N-(2-ethyl-6-methylphenyl)-2-[3-(2-methoxy-5-nitrophenyl)-thioureido]-2-phenyl acetamide was isolated as a yellow solid (22.3 mg, 78%) from 2-Amino-N-(2-ethyl-6-methylphenyl)-2-phenylacetamide (15.8 mg, 0.06 mmol) and 2-methoxy-5-nitrophenylisothiocyanate (14.9 mg, 0.07 mmol).

Experiment 9:

C9 tert-butyl [1-(2-isopropyl-6-methylphenylcarbamoyl)-1-phenyl-methyl]-carbamate

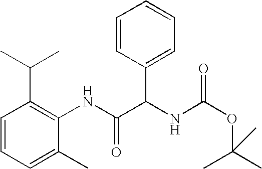

tert-butyl [1-(2-isopropyl-6-methylphenylcarbamoyl)-1-phenyl-methyl]-carbamate was isolated as a white solid (216.1 mg, 57%) from BOC-phenylglycine (250.0 mg, 0.99 mmol), N-methylmorpholine (0.12 mL, 1.09 mmol), isobutylchloroformate (0.14 mL, 1.09 mmol), 2-isopropyl-6-methylaniline (0.19 mL, 1.19 mmol) and N-methylmorpholine (0.13 mL, 1.19 mmol).

D9 2-Amino-N-(2-isopropyl-6-methylphenyl)-2-phenylacetamide

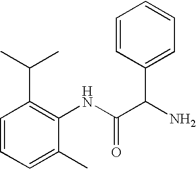

2-Amino-N-(2-isopropyl-6-methylphenyl)-2-phenylacetamide was isolated as a white solid (78.8 mg, 51%) from tert-butyl [1-(2-isopropyl-6-methylphenylcarbamoyl)-1-phenyl-methyl]-carbamate (210.0 mg, 0.55 mmol).

E9.1 N-(2-isopropyl-6-methylphenyl)-2-[3-(2-methoxy-5-nitrophenyl)-thioureido]-2-phenyl acetamide

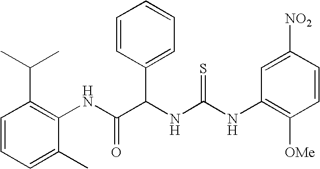

N-(2-isopropyl-6-methylphenyl)-2-[3-(2-methoxy-5-nitrophenyl)-thioureido]-2-phenyl acetamide was isolated as a yellow solid (15.3 mg, 52%) from 2-Amino-N-(2-isopropyl-6-methylphenyl)-2-phenylacetamide (16.7 mg, 0.06 mmol) and 2-methoxy-5-nitrophenylisothiocyanate (14.9 mg, 0.07 mmol).

Experiment 10:

C10 tert-butyl [1-(2-chloro-6-methylphenylcarbamoyl)-1-phenyl-methyl]-carbamate

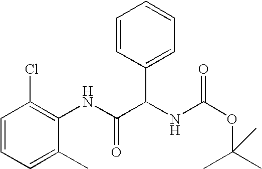

tert-butyl [1-(2-chloro-6-methylphenylcarbamoyl)-1-phenyl-methyl]-carbamate was isolated as a white solid (139.2 mg, 38%) from BOC-phenylglycine (250.0 mg, 0.99 mmol), N-methylmorpholine (0.12 mL, 1.09 mmol), isobutylchloroformate (0.14 mL, 1.09 mmol), 2-chloro-6-methylaniline (0.15 mL, 1.19 mmol) and N-methylmorpholine (0.13 mL, 1.19 mmol).

D10 2-Amino-N-(2-chloro-6-methylphenyl)-2-phenylacetamide

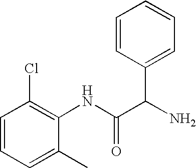

2-Amino-N-(2-chloro-6-methylphenyl)-2-phenylacetamide was isolated as a white solid (37.9 mg, 38%) from tert-butyl [1-(2-chloro-6-methylphenylcarbamoyl)-1-phenyl-methyl]-carbamate (132.8 mg, 0.36 mmol).

E10.1 N-(2-chloro-6-methylphenyl)-2-[3-(2-methoxy-5-nitrophenyl)-thioureido]-2-phenyl acetamide

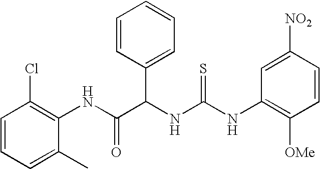

acetamide was isolated as a yellow solid (14.8 mg, 76%) from 2-Amino-N-(2-chloro-6-methylphenyl)-2-phenylacetamide (10.0 mg, 0.04 mmol) and 2-methoxy-5-nitrophenyl isothiocyanate (9.2 mg, 0.04 mmol).

Experiment 11:

C11 tert-butyl [1-(2,4-dimethylphenylcarbamoyl)-1-phenyl-methyl]-carbamate

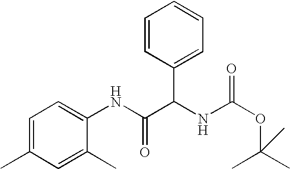

tert-butyl [1-(2,4-dimethylphenylcarbamoyl)-1-phenyl-methyl]-carbamate was isolated as a white solid (240.2 mg, 68%) from BOC-phenylglycine (250.0 mg, 0.99 mmol), N-methylmorpholine (0.12 mL, 1.09 mmol), isobutylchloroformate (0.14 mL, 1.09 mmol), 2,4-dimethylaniline (0.15 mL, 1.19 mmol) and N-methylmorpholine (0.13 mL, 1.19 -mmol).

D11.1 2-Amino-N-(2,4-dimethylphenyl)-2-phenylacetamide

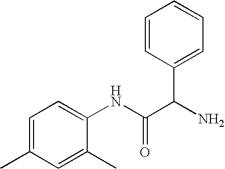

2-Amino-N-(2,4-dimethylphenyl)-2-phenylacetamide was isolated as a white solid (86.1 mg, 52%) from tert-butyl [1-(2,4-dimethylphenylcarbamoyl)-1-phenyl-methyl]-carbamate (231.0 mg, 0.65 mmol).

E11 N-(2,4-dimethylphenyl)-2-[3-(2-methoxy-5-nitrophenyl)-thioureido]-2-phenyl acetamide

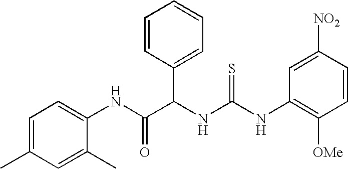

N-(2,4-dimethylphenyl)-2-[3-(2-methoxy-5-nitrophenyl)-thioureido]-2-phenyl acetamide was isolated as a yellow solid (21.4 mg, 77%) from 2-Amino-N-(2,4-dimethylphenyl)-2-phenylacetamide (15.0 mg, 0.06 mmol) and 2-methoxy-5-nitrophenylisothiocyanate (7.9 mg, 0.038 mmol).

Experiment 12:

C12 tert-butyl [1-(2,6-dimethylphenylcarbamoyl)-1-phenyl-methyl]-carbamate

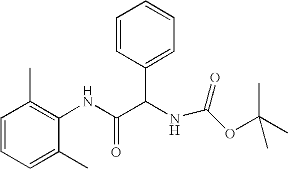

tert-butyl [1-(2,6-dimethylphenylcarbamoyl)-1-phenyl-methyl]-carbamate was isolated (1.61 g, 76%) from BOC-phenyl glycine (1.50 g, 5.97 mmol), N-methylmorpholine (0.72 mL, 6.57 mmol), isobutylchloroformate (0.85 mL, 6.57 mmol), 2,6-dimethylaniline (0.88 mL, 7.16 mmol) and N-methylmorpholine (0.79 mL, 7.16 mmol).

D12 2-Amino-N-(2,6-dimethylphenyl)-2-phenylacetamide

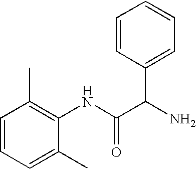

2-Amino-N-(2,6-dimethylphenyl)-2-phenylacetamide was isolated as a white solid (717.2 mg, 67%) from tert-butyl [1-(2,6-dimethylphenylcarbamoyl)-1-phenyl-methyl]-carbamate (1.50 g, 4.24 mmol).

4-chloro-3-nitro-(N-methyl)piperizinylphenylsulphonamide

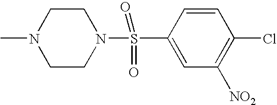

4-chloro-3-nitro-(N-methyl)piperizinylsulphonamide was isolated as a light yellow solid (474.3 mg, 76%) from 4-chloro-3-nitrobenzene sulphonyl chloride (500.0 mg, 1.95 mmol) and diisopropylethylamine (0.34 mL, 1.95 mmol) and N-methylpiperizine (0.22 mL, 1.95 mmol).

4-amino-3-nitro-(N-methyl)piperizinylphenylsulphonamide

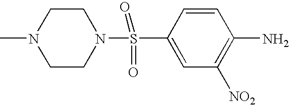

4-amino-3-nitro-(N-methyl) piperizinylsulphonamide was isolated as a yellow solid (14.2 mg, 14%) from 4-chloro-3-nitro-(N-methyl)piperizinylphenylsulphonamide (110.5 mg, 0.35 mmol) and 2M NH₃/MeOH (5 mL).

1 -(N-methyl)piperizinyl sulphonamido-3-nitro-4-phenylisothiocyanate

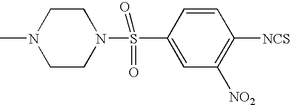

1-(N-methyl)piperizinyl sulphonamido-3-nitro-4-phenylisothiocyanate was isolated as a clear yellow oil (1.8 mg, 13%) from 4-amino-3-nitro-(N-methyl) piperizinylphenylsulphonamide (13.2 mg, 0.04 mmol) and DPT (10.2 mg, 0.04 mmol).

E12.1 N-(2,6-dimethylphenyl)-2-[3-(2-methyl-4-(N-methyl)piperizinyl sulphonamidophenyl)-thioureido]-2-phenyl acetamide

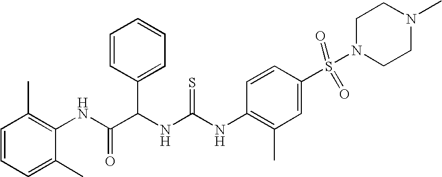

N-(2,6-dimethylphenyl)-2-[3-(2-methyl-4-(N-methyl)piperizinyl suiphonamido phenyl)-thioureido]-2-phenyl acetamide was isolated (3.1 mg, 103%) from 2-Amino-N-(2,6-dimethylphenyl)-2-phenylacetamide (1.3 mg, 0.005 mmol) and 1-(N-methyl) piperizinyl sulphonamido-3-nitro-4-phenylisothiocyanate (1.8 mg, 0.005 mmol).

Experiment 13:

C13 tert-butyl [1-(4-methylphenylcarbamoyl)-1-phenyl-methyl]-carbamate

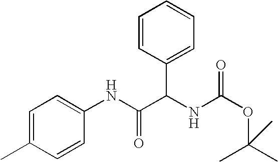

tert-butyl [1-(4-methylphenylcarbamoyl)-1-phenyl-methyl]-carbamate was isolated as a white solid (1.88 g, 139%) from [(tert-butoxycarbonyl)amino](phenyl)acetic acid (1.0 g, 3.98 mmol), N-methylmorpholine (0.48 mL, 4.38 mmol), isobutylchloroformate (0.57 mL, 4.38 mmol) and 4-methylaniline (0.51 g, 4.78 mmol)and N-methylmorpholine (0.52 mL, 4.78 mmol).

D13 2-Amino-N-(4-methylphenyl)-2-phenylacetamide

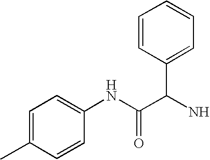

To a 50 mL round bottom flask equipped with a stirring bar was added tert-butyl [1-(4-methylphenylcarbamoyl)-1-phenyl-methyl]-carbamate (1.88 g, 5.52 mmol) and formic acid (15 mL). The reaction was stirred at 50° C. for 1.5 hours after which it was cooled to room temperature. The formic acid was removed in vacuo and the resulting oil was taken up into EtOAc (75 mL) and water (75 mL). 1 N NaOH was added until the pH was 8-9 and the aqueous layer was extracted with EtOAc two additional times. The combined organic layers were washed with brine, dried over MgSO₄, filtered and concentrated. The crude product was purified by column chromatography (Hexanes:EtOAc 60:40 to CH₂Cl₂:MeOH 95:5 to yield the title compound as an off white solid (874.2 mg, 91%).

E13.1 N-(4-methylphenyl)-2-[3-(1-naphthyl)-thioureido]-2-phenyl acetamide

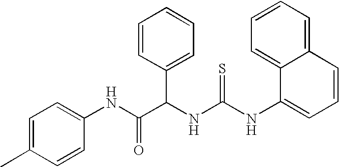

N-(4-methylphenyl)-2-[3-(1-naphthyl)-thioureido]-2-phenyl acetamide was isolated as a white solid (37.4 mg, 70%) from 2-Amino-N-(4-methylphenyl)-2-phenylacetamide (30.3 mg, 0.13 mmol) and 1-naphthylisothiocyanate (35.0 mg, 0.19 mmol).

E13.2 N-(2-(4-methylphenyl)-2-[3-(2-methoxy-5-nitrophenyl)-thioureido]-2-phenyl acetamide

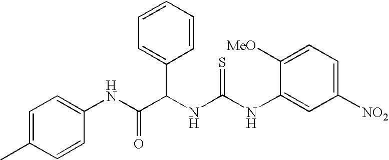

N-(2-(4-methylphenyl)-2-[3-(2-methoxy-5-nitrophenyl)-thioureido]-2-phenyl acetamide was isolated (21.0 mg, 38%) from 2-amino-N-(2-isopropylphenyl)-2-phenylacetamide (30.0 mg, 0.124 mmol) and 2-methoxy-5-nitrophenylisothiocyanate (31.5 mg, 0.145 mmol).

E13.3 2-[3-(2-chloro-5-nitrophenyl)-thioureido]-N-(4-methylphenyl)-2-phenyl acetamide

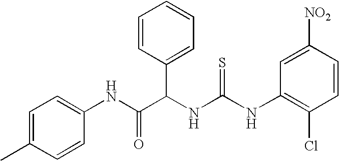

2-[3-(2-chloro-5-nitrophenyl)-thioureido]-N-(4-methylphenyl)-2-phenyl acetamide (22.6 mg, 99%); from 2-Amino-N-(4-methylphenyl)-2-phenylacetamide (17 mg, 0.05 mmoles) reacted with 2-chloro-5-nitrophenylisothiocyanate (16.3, 0.075 mmoles) in dichloromethane (0.8 ml) 60° C.

E13.4 2-[3-(2-methoxy-5-methylphenyl)-thioureido]-N-(4-methylphenyl)-2-phenyl acetamide

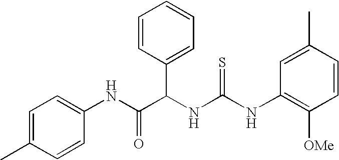

2-[3-(2-methoxy-5-methylphenyl)-thioureido]-N-(4-methylphenyl)-2-phenyl acetamide (15.7 mg, 74.8%); from 2-Amino-N-(4-methylphenyl)-2-phenylacetamide (17 mg, 0.05 mmoles) reacted with 2-methoxy-5-methylphenylisothiocyanate (13.4, 0.075 mmoles) in dichloromethane (0.8 ml) at 60° C.

E13.5 2-[3-(5-chloro-2-methoxy-phenyl)-thioureido]-N-(4-methylphenyl)-2-phenyl acetamide

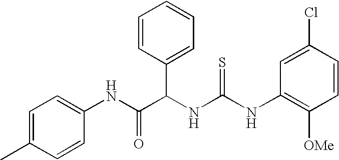

2-[3-(5-chloro-2-methoxy-phenyl)-thioureido]-N-(4-methylphenyl)-2-phenyl acetamide (20.4 mg, 92.7%); from 2-Amino-N-(4-methylphenyl)-2-phenylacetamide (17 mg, 0.05 mmoles) reacted with 5-chloro-2-methoxy-phenylisothiocyanate (15, 0.075 mmoles) in dichloromethane (0.8 ml) at 60° C.

E13.6 2-[3-(2,5-dimethoxy-phenyl)-thioureido]-N-(4-methylphenyl)-2-phenyl acetamide

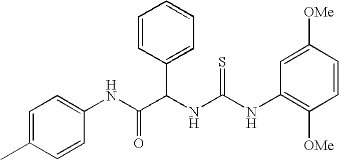

2-[3-(2,5-dimethoxy-phenyl)-thioureido]-N-(4-methylphenyl)-2-phenyl acetamide (22.4 mg, 72.4%); from 2-Amino-N-(4-methylphenyl)-2-phenylacetamide (17 mg, 0.05 mmoles) reacted with 2,5-dimethoxy-phenylisothiocyanate (14.64, 0.075 mmoles) in dichloromethane (0.8 ml) at 60° C.

E13.7 2-[3-( 2-methoxy-5-nitro-phenyl)-ureido]-N-(4-methylphenyl)-2-phenyl acetamide

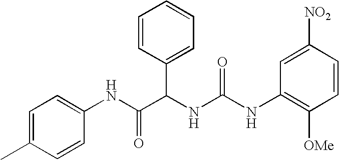

2-[3-(2-methoxy-5-nitro-phenyl)-ureido]-N-(4-methylphenyl)-2-phenyl acetamide (53.3 mg, 87.3%); from 2-Amino-N-(4-methylphenyl)-2-phenylacetamide (34 mg, 0.1 mmoles) reacted with 2-methoxy-5-nitro-phenylisocyanate (23.3 mg, 0.12 mmoles) in dichloromethane (1.2 ml) at 60° C.

E13.8 (R)-2-[3-(2-methoxy-5-nitro-phenyl)-ureido]-N-(4-methylphenyl)-2-phenyl acetamide and (S)-2-[3-(2-methoxy-5-nitro-phenyl)-ureido]-N-(4-methylphenyl)-2-phenyl acetamide

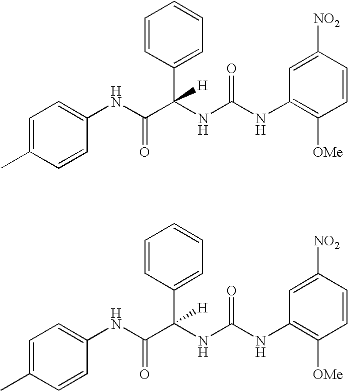

(R)-2-[3-(2-methoxy-5-nitro-phenyl)-ureido]-N-(4-methylphenyl)-2-phenyl acetamide and (S)-2-[3-(2-methoxy-5-nitro-phenyl)-ureido]-N-(4-methylphenyl)-2-phenyl acetamide were separated from 2-[3-(2-methoxy-5-nitro-phenyl)-ureido]-N-(4-methylphenyl)-2-phenyl acetamide by chiracel OD column with HPLC [ethanol(1% acetic acid): Hexane=20:80]

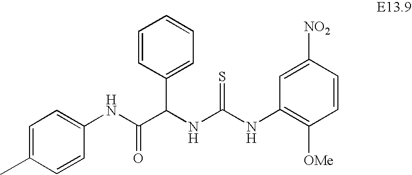

Experiment 14:

C14 tert-butyl [1-(phenylcarbamoyl)-1-phenyl-methyl]-carbamate

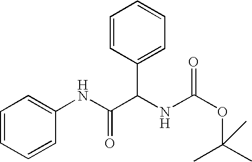

tert-butyl [1-(phenylcarbamoyl)-1-phenyl-methyl]-carbamate was isolated as a white solid (1.42 g, 110%) from [(tert-butoxycarbonyl)amino](phenyl)acetic acid (1.0 g, 3.98 mmol), N-methylmorpholine (0.48 mL, 4.38 mmol), isobutylchloroformate (0.57 mL, 4.38 mmol), aniline (0.44 mL, 4.78 mmol) and N-methylmorpholine (0.52 mL, 4.78 mmol).

D14 2-Amino-N-(phenyl)-2-phenylacetamide

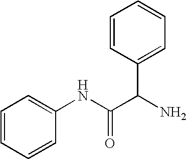

2-Amino-N-(phenyl)-2-phenylacetamide was isolated as a white solid (754.5 mg, 84%) from tert-butyl [1-(phenylcarbamoyl)-1-phenyl-methyl]-carbamate (1.42 g, 4.36 mmol).

E14.1 N-(Phenyl)-2-[3-(2-methoxyphenyl)-thioureido]-2-phenyl acetamide

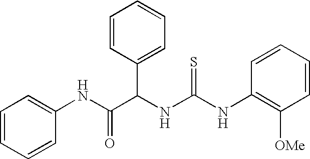

N-(phenyl)-2-[3-(2-methoxyphenyl)-thioureido]-2-phenyl acetamide was isolated as a white solid (28.9 mg, 56%) from 2-Amino-N-(phenyl)-2-phenylacetamide (29.7 mg, 0.13 mmol) and 2-methoxyphenylisothiocyanate (35.0 mg, 0.20 mmol).

E14.2 N-(Phenyl)-2-[3-(2-fluorophenyl)-thioureido]-2-phenyl acetamide

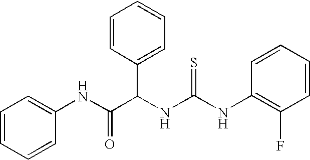

N-(phenyl)-2-[3-(2-fluorophenyl)-thioureido]-2-phenyl acetamide was isolated as a white solid (38.7 mg, 72%) from 2-Amino-N-(phenyl)-2-phenylacetamide (32.2 mg, 0.14 mmol) and 2-fluorophenylisothiocyanate (33.0 mg, 0.21 mmol).

Experiment 15:

C15 tert-butyl [1-(3-methylphenylcarbamoyl)-1-phenyl-methyl]-carbamate

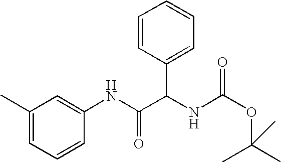

tert-butyl [1-(3-methylphenylcarbamoyl)-1-phenyl-methyl]-carbamate was isolated as an off-white solid (1.46 g, 108%) from [(tert-butoxycarbonyl)amino](phenyl)acetic acid (1.0 g, 3.98 mmol), N-methylmorpholine (0.48 mL, 4.38 mmol), isobutylchloroformate (0.57 mL, 4.38 mmol), 3-methylaniline (0.52 mL, 4.78 mmol) and N-methylmorpholine (0.52 mL, 4.78 mmol).

D15 2-Amino-N-(3-methylphenyl)-2-phenylacetamide

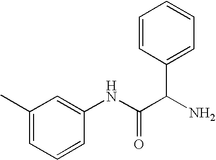

2-Amino-N-(3-methylphenyl)-2-phenylacetamide was isolated as an off-white solid (917.1 mg, 96%) from tert-butyl [1-(3-methylphenylcarbamoyl)-1-phenyl-methyl]-carbamate (1.46 g, 4.29 mmol).

E15.1 N-(3-methylphenyl)-2-[3-(2-fluorophenyl)-thioureido]-2-phenyl acetamide

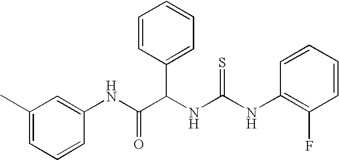

N-(3-methylphenyl)-2-[3-(2-fluorophenyl)-thioureido]-2-phenyl acetamide was isolated as pale yellow solid (31.6 mg, 63%) from 2-Amino-N-(3-methylphenyl)-2-phenylacetamide (30.4 mg, 0.13 mmol) and 2-fluorophenylisothiocyanate (29.0 mg, 0.19 mmol).

E15.2 N-(3-methylphenyl)-2-[3-(2-methoxyphenyl)-thioureido]-2-phenyl acetamide

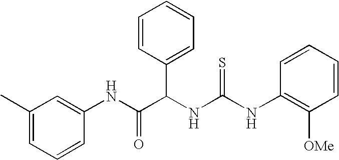

N-(3-methylphenyl)-2-[3-(2-methoxyphenyl)-thioureido]-2-phenyl acetamide was isolated as a white solid (19.7 mg, 38%) from 2-Amino-N-(3-methylphenyl)-2-phenylacetamide (30.9 mg, 0.13 mmol) and 2-methoxyphenylisothiocyanate (34.5 mg, 0.19 mmol).

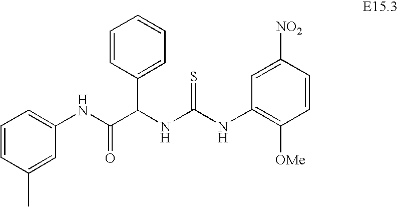

Experiment 16:

E16.1 N-(2-(2-isopropylphenyl)-2-[3-(2-methoxyphenyl)-thioureido]-2-phenyl acetamide

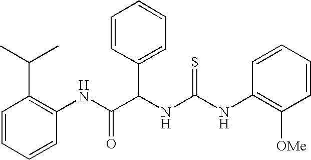

N-(2-(2-isopropylphenyl)-2-[3-(2-methoxyphenyl)-thioureido]-2-phenyl acetamide was isolated (13.0 mg, 27%) from 2-amino-N-(2-isopropylphenyl)-2-phenylacetamide (30.0 mg, 0.111 mmol) and 2-methoxyphenylisothiocyanate (30.0 mg, 0.166 mmol).

Experiment 17:

E17.1 N-(2-(4-trifluoromethylphenyl)-2-[3-(2-methoxy-5-nitrophenyl)-thioureido]-2-(2,3-difluoro)phenyl acetamide

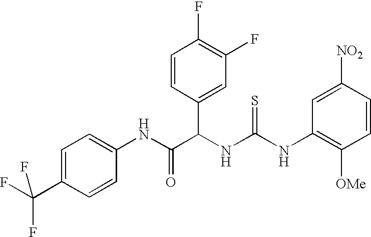

N-(2-(4-trifluoromethylphenyl)-2-[3-(2-methoxy-5-nitrophenyl)-thioureido]-2-(2,3-difluoro)phenyl acetamide was isolated (17.7 mg, 60%) from 2-amino-N-(4-trifluoromethylphenyl)-2-(3,4-difluoro) phenylacetamide (20.0 mg, 0.061 mmol) and 2-methoxy-5-nitroisothiocyanate (19.0 mg, 0.090 mmol).

Experiment 18:

E18.1 N-(2-(4-trifluoromethylphenyl)-2-[3-(2-methoxy-5-nitrophenyl)-thioureido]-2-(3-trifluoromethyl)phenyl acetamide

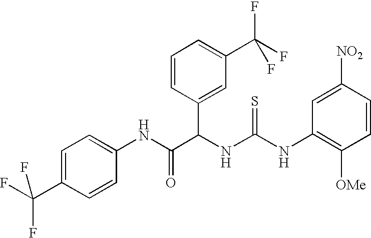

N-(2-(4-trifluoromethylphenyl)-2-[3-(2-methoxy-5-nitrophenyl)-thioureido]-2-(3-trifluoromethyl)phenyl acetamide was isolated (20.0 mg, 81%) from 2-amino-N-(4-trifluoromethylphenyl)-2-(3-trifluoromethyl) phenylacetamide (20.0 mg, 0.055 mmol) and 2-methoxy-5-nitrophenylisothiocyanate (18.0 mg, 0.082 mmol).

Experiment 19:

C19 tert-butyl [1-(4-isopropylphenylcarbamoyl)-1-phenyl-methyl]-carbamate

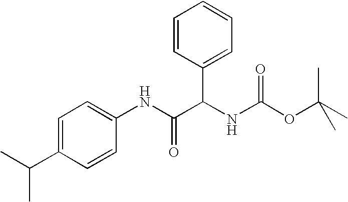

To the mixture of [(tert-butoxycarbonyl)amino](phenyl)acetic acid (500 mg, 2 mmoles) and N-methylmorpholine (241 μl, 2.2 mmoles) in THF (5 ml) at −40˜−50° C. was added isobutyl chloroformate (235 μl, 2.2 mmoles). After the reaction mixture was stirred for 2 hours, a mixture of 4-isopropylaniline (325 mg, 2.4 mmoles) and N-methylmorpholine (263 ml, 2.4 mmoles) were added. After the reaction mixture was stirred and left overnight, it was diluted with dichloromethane (20 ml) and washed with water (20 ml), 1 M sodium hydrosulphate (20 ml X 3) and brine (20 ml), dried with sodium sulfate, concentrated. The residue was triturated with hexanes to give the white solid tert-butyl [1-(4-isopropylphenylcarbamoyl)-1-phenyl-methyl]-carbamate (577 mg, yield 78.7%).

D19 2-Amino-N-(4-isopropylphenyl)-2-phenylacetamide formic acid salt

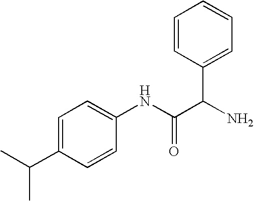

tert-butyl [1-(4-isopropylphenylcarbamoyl)-1-phenyl-methyl]-carbamate (500 mg, 1.36 mmoles) was mixed with 96% formic acid (5 ml) and heated to 60° C. for 0.5 hour. The reaction mixture was concentrated by evaporation. The residue was triturated with hexanes and ether (1:1, 10 ml) to give the white solid product, 2-Amino-N-(4-isopropylphenyl)-2-phenylacetamide formic acid salt (426 mg, quantitative).

E19.1 N-(4-isopropylphenyl)-2-[3-(2-methoxy-5-nitrophenyl)-thioureido]-2-phenyl acetamide

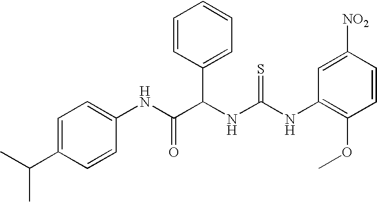

2-Amino-N-(4-isopropylphenyl)-2-phenylacetamide formic acid salt (15.6 mg, 0.05 mmoles) was mixed with 2-methoxy-5-nitrophenylthioisocyanate (12.6 mg, 0.06 mmoles) and triethylamine (15.2 mg, 0.15 mmoles) in dichloromethane (1 ml) at ambient temperature. The reaction was stirred overnight. The solvent was removed by evaporation, and the product was purified by column chromatography with 15% ethyl acetate in hexanes and 30% ethyl acetate in hexanes to give N-(4-isopropylphenyl)-2-[3-(2-methoxy-5-nitrophenyl)-thioureido]-2-phenyl acetamide (18.8 mg, yield 79.2%)

Experiment 20:

C20 tert-butyl [1-(4-trifluoromethylphenylcarbamoyl)-1-phenyl-methyl]-carbamate

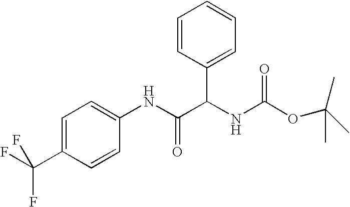

tert-butyl [1-(4-trifluoromethylphenylcarbamoyl)-1-phenyl-methyl]-carbamate (708 mg, 96.5%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being queched with 4-trifluoromethylaniline (386 mg, 2.4 mmoles).

D20 2-Amino-N-(4-trifluoromethyphenyl)-2-phenylacetamide

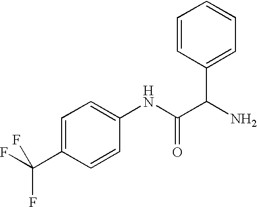

2-Amino-N-(4-trifluoromethylphenyl)-2-phenylacetamide (375 mg, quantitative); from tert-butyl [1-(4-trifluoromethylphenylcarbamoyl)-1-phenyl-methyl]-carbamate (500 mg, 1.27 mmoles) reacted with formic acid (5 ml) at 60° C. for 0.5 hour.

E20.1 N-(4-trifluoromethylphenyl)-2-[3-(2-methoxy-5-nitrophenyl)-thioureido]-2-phenyl acetamide

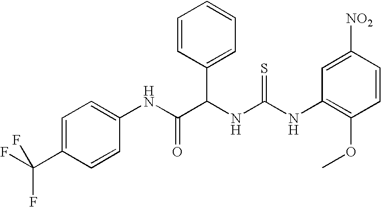

N-(4-trifluoromethylphenyl)-2-[3-(2-methoxy-5-nitrophenyl)-thioureido]-2-phenyl acetamide(19.2 mg, 76.2%); from 2-Amino-N-(4-trifluoromethyphenyl)-2-phenylacetamide (14.7 0.05 mmoles) reacted with 2-methoxy-5-nitrophenylisothiocyanate (12.6 mg, 0.06 mmoles) and triethylamine (15.2 mg, 0.15 mmoles) in dichloromethane (1 ml) at ambient temperature overnight.

E20.2 2-[3-(2-chloro-5-nitrophenyl)-thioureido]-N-(4-trifluoromethylphenyl)-2-phenyl acetamide

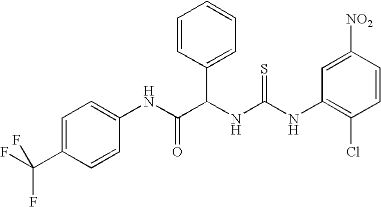

2-[3-(2-chloro-5-nitrophenyl)-thioureido]-N-(4-trifluoromethylphenyl)-2-phenyl acetamide (12.5 mg, 74%); from 2-Amino-N-(4-trifluoromethylphenyl)-2-phenylacetamide (13.1 mg, 0.0332 mmoles) reacted with 2-chloro-5-nitrophenylisothiocyanate (9.7 mg, 0.045 mmoles) in dichloromethane (1 ml) at 60° C. overnight.

E20.3 2-[3-(5-chloro-2-methoxyphenyl)-thioureido]-N-(4-trifluoromethylphenyl)-2-phenyl acetamide

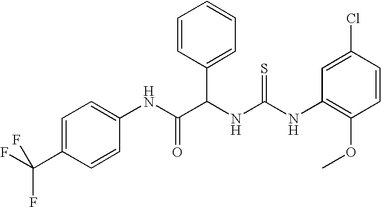

2-[3-(5-chloro-2-methoxyphenyl)-thioureido]-N-(4-trifluoromethylphenyl)-2-phenyl acetamide (13.5 mg, 82.3%); from 2-Amino-N-(4-trifluoromethylphenyl)-2-phenylacetamide (13.1 mg, 0.0332 mmoles) reacted with 5-chloro-2-methoxyphenylthioisocyanate (9.0 mg, 0.045 mmoles) in dichloromethane (1 ml) 15 at 60° C. overnight.

E20.4 2-[3-(2-methoxy-5-nitro-phenyl)-ureido]-N-(4-trifluoromethylphenyl)-2-phenyl acetamide

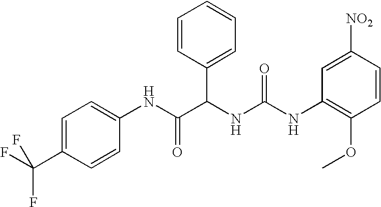

2-[3-(2-methoxy-5-nitro-phenyl)-ureido]-N-(4-trifluoromethylphenyl)-2-phenyl acetamide (16.1 mg, 64.6%); from 2-Amino-N-(4-trifluoromethylphenyl)-2-phenylacetamide (15 mg, 0.051 mmoles) reacted with 2-methoxy-5-nitrophenylisocyanate (15 mg, 0.077 mmoles) in dichloromethane (0.8 ml) at 60° C. overnight.

Experiment 21:

E21.1 2-[3-(2-chloro-5-nitro-phenyl)-thioureido]-N-(3-trifluoromethylphenyl)-2-phenyl acetamide

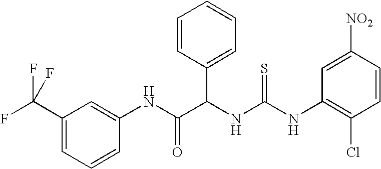

2-[3-(2-chloro-5-nitro-phenyl)-thioureido]-N-(3-trifluoromethylphenyl)-2-phenyl acetamide (23.5 mg, 90.5%); from 2-Amino-N-(3-trifluoromethylphenyl)-2-phenylacetamide (15 mg, 0.051 mmoles) reacted with 2-chloro-5-nitrophenylisothiocyanate (15 mg, 0.07 mmoles) in dichloromethane (0.5 ml) at 60° C. overnight.

Experiment 22:

C22 tert-butyl [1-(8-quinolinylcarbamoyl)-1-phenyl-methyl]-carbamate

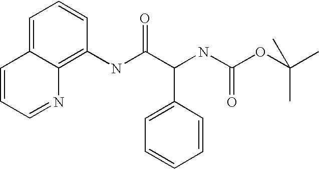

tert-butyl [1-(8-quinolinylcarbamoyl)-1-phenyl-methyl]-carbamate (1.696 g); was made from [(tert-butoxycarbonyl)amino](phenyl)acetic acid (1.0 g, 3.98 mmoles) reacted with isobutyl chloroformate (569.4,μl, 4.38 mmoles) and N-methylmorpholine (482.2 μl, 4.38 mmoles) in THF (10 ml) at −40˜−50° C., followed by being queched with 8-aminoquinoline (689.2 mg, 4.78 mmoles).

D22 2-Amino-N-(8-quinolinyl)-2-phenylacetamide formic acid salt

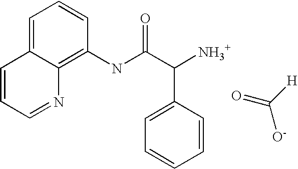

2-Amino-N-(8-quinolinyl)-2-phenylacetamide formic acid salt (0.915 g, quantitative); from tert-butyl [1-(8-quinolinylcarbamoyl)-1-phenyl-methyl]-carbamate (1.1 g, 2.9 mmoles) reacted with formic acid (20 ml) at 60° C. for 0.5 hour.

E22.1 2-[3-(2-methoxy-5-nitro-phenyl)-ureido]-N-(8-quinolinyl)-2-phenyl acetamide

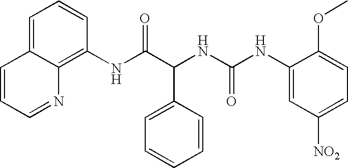

2-[3-(2-methoxy-5-nitro-phenyl)-ureido]-N-(8-quinolinyl)-2-phenyl acetamide (17 mg, 66.7%); from 2-Amino-N-(8-quinolinyl)-2-phenylacetamide (15 mg, 0.054 mmoles) reacted with 2-methoxy-5-nitrophenylisocyanate (15 mg, 0.077 mmoles) in dichloromethane (0.8 ml) at 60° C. overnight.

E22.2 2-[3-(2-methoxy-5-nitro-phenyl)-thioureido]-N-(8-quinolinyl)-2-phenyl acetamide

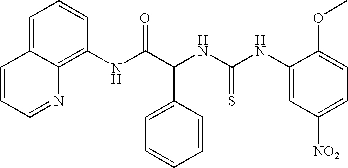

2-[3-(2-methoxy-5-nitro-phenyl)-thioureido]-N-(8-quinolinyl)-2-phenyl acetamide (14.6 mg, 83.2%); from 2-Amino-N-(8-quinolinyl)-2-phenylacetamide (10 mg, 0.036 mmoles) reacted with 2-methoxy-5-nitrophenylisothiocyanate (10 mg, 0.0476 mmoles) in dichloromethane (0.5 ml) at 60° C. overnight.

E22.3 2-[3-(2-methoxy-5-nitro-phenyl)-ureido]-N-(6-quinolinyl)-2-phenyl acetamide

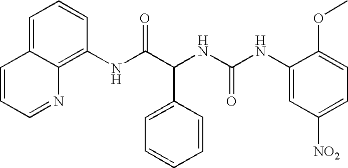

2-[3-(2-methoxy-5-nitro-phenyl)-ureido]-N-(8-quinolinyl)-2-phenyl acetamide (17 mg, quantitative); from 2-Amino-N-(8-quinolinyl)-2-phenylacetamide (10 mg, 0.036 mmoles) reacted with 2-methoxy-5-nitrophenylisocyanate (9 mg, 0.046 mmoles) in dichloromethane (0.5 ml) at 60° C. overnight.

Experiment 23:

C23 tert-butyl [1-(4-methyl-3-trifluoromethylphenylcarbamoyl)-1-phenyl-methyl]-carbamate

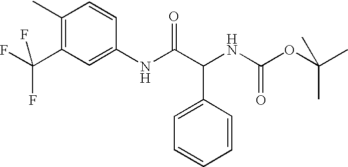

tert-butyl [1-(4-methyl-3-trifluoromethylphenylcarbamoyl)-1-phenyl-methyl]-carbamate (762 mg, 93.7%); from [(tert-butoxycarbonyl)amino](phenyl)acetic acid (500 mg, 2 mmoles) reacted with isobutyl chloroformate (235,μl, 2.2 mmoles) and N-methylmorpholine (241 μl, 2.2 mmoles) in THF (5 ml) at −40˜−50° C., followed by being queched with 4-methy-3-trifluoromethylaniline (420.4 mg, 2.4 mmoles).

D23 2-Amino-N-(4-methyl-3-trifluoromethyphenyl)-2-phenylacetamide

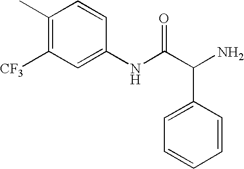

2-Amino-N-(4-methyl-3-trifluoromethyphenyl)-2-phenylacetamide (409.7 mg, 71.4%); from tert-butyl [1-(4-methyl-3-trifluoromethylphenylcarbamoyl)-1-phenyl-methyl]-carbamate (760 mg, 1.86 mmoles) reacted with formic acid (4 ml) at 60° C. for 0.5 hour.

E23.1 N-(4-methyl-3-trifluoromethylphenyl)-2-[3-(2-methoxy-5-nitrophenyl)-thioureido]-2-phenyl acetamide

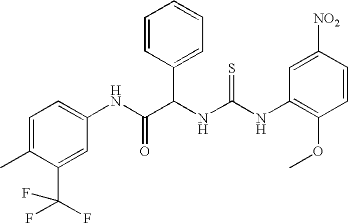

N-(4-methyl-3-trifluoromethylphenyl)-2-[3-(2-methoxy-5-nitrophenyl)-thioureido]-2-phenyl acetamide(8.0 mg, 48.2%); from 2-Amino-N-(4-methyl-3-trifluoromethyphenyl)-2-phenylacetamide (10 mg, 0.032 mmoles) reacted with 2-methoxy-5-nitrophenylisothiocyanate (9.0 mg, 0.043 mmoles) in dichloromethane (1 ml) at ambient temperature overnight.

E23.2 N-(4-methyl-3-trifluoromethylphenyl)-2-[3-(2-methoxy-5-nitrophenyl)-ureido]-2-phenyl acetamide

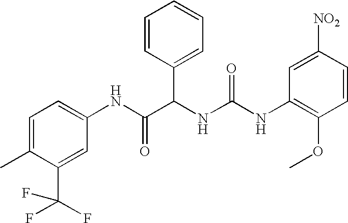

N-(4-methyl-3-trifluoromethylphenyl)-2-[3-(2-methoxy-5-nitrophenyl)-ureido]-2-phenyl acetamide (14.2 mg, 88.3%); from 2-Amino-N-(4-methyl-3-trifluoromethyphenyl)-2-phenylacetamide (10 mg, 0.032 mmoles) reacted with 2-methoxy-5-nitrophenylisocyanate (9.0 mg, 0.046 mmoles) in dichloromethane (1 ml) 60° C. overnight.

Experiment 24:

C24 tert-butyl [1-(3-dim thylamino-phenylcarbamoyl)-1-phenyl-methyl]-carbamate

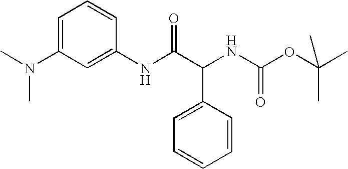

tert-butyl [1-(3-dimethylamino-phenylcarbamoyl)-1-phenyl-methyl]-carbamate (241 mg, 32.8%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2,2 mmoles) in THF (5 ml) at −40˜−50° C., followed by being queched with 3-dimethylaminoaniline hydrochloride (501.88 mg, 2.4 mmoles).

D24 2-Amino-N-(3-N,N-dimethylaminophenyl)-2-phenylacetamide

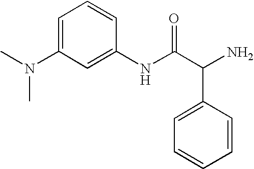

2-Amino-N-(3-N,N-dimethylaminophenyl)-2-phenylacetamide (25.4 mg, 14.1%); from tert-butyl [1-(3-dimethylamino-phenylcarbamoyl)-1-phenyl-methyl]-carbamate (247 mg, 0.669 mmoles) reacted with formic acid (1.5 ml) at 60° C. for 0.5 hour.

E24.1 N-(3-N,N-dimethylphenyl)-2-[3-(2-methoxy-5-nitrophenyl)-thioureido]-2-phenyl acetamide

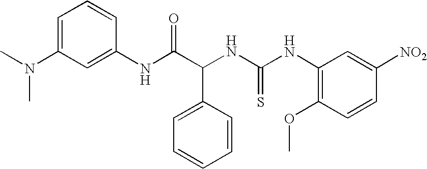

N-(3-N,N-dimethylphenyl)-2-[3-(2-methoxy-5-nitrophenyl)-thioureido]-2-phenyl acetamide (11.8 mg, 82%); from 2-Amino-N-(4-methyl-3-trifluoromethyphenyl)-2-phenylacetamide (8.2 mg, 0.042 mmoles) reacted with 2-methoxy-5-nitrophenylisothiocyanate (9.0 mg, 0.043 mmoles) in dichloromethane (1 ml) at 60° C. overnight.

E24.2 N-(3-N,N-dimethylphenyl)-2-[3-(2-methoxy-5-nitrophenyl)-ureido]-2-phenyl acetamide

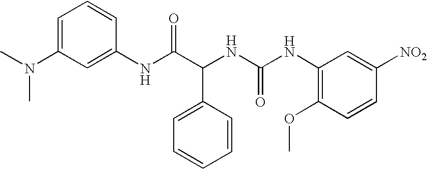

N-(3-N,N-dimethyl phenyl)-2-[3-(2-methoxy-5-nitrophenyl)-ureido]-2-phenyl acetamide (9.3 mg, 66.2%); from 2-Amino-N-(4-methyl-3-trifluoromethyphenyl)-2-phenylacetamide (8.2 mg, 0.042 mmoles) reacted with 2-methoxy-5-nitrophenylisocyanate (9.0 mg, 0.046 mmoles) in dichloromethane (1 ml) 60° C. overnight.

Experiment 25:

C25 tert-butyl [1-(6-quinolinylcarbamoyl)-1-phenyl-methyl]-carbamate

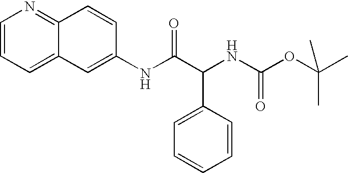

tert-butyl [1-(6-quinolinylcarbamoyl)-1-phenyl-methyl]-carbamate (177 mg, 24.6%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being queched with 6-aminoquinoline (346 mg, 2.4 mmoles).

D25 2-Amino-N-(6-quinolinyl)-2-phenylacetamide

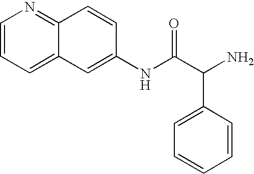

2-Amino-N-(6-quinolinyl)-2-phenylacetamide (62 mg, 47.6); from tert-butyl [1-(6-quinolinylcarbamoyl)-1-phenyl-methyl]-carbamate (177 mg, 0.469 mmoles) reacted with formic acid (1 ml) at 60° C. for 0.5 hour.

E25.1 N-(quinolin-6-yl)-2-[3-(2-methoxy-5-nitrophenyl)-thioureido]-2-phenyl acetamide

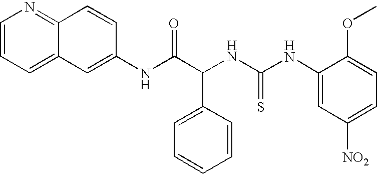

N-(quinolin-6-yl)-2-[3-(2-methoxy-5-nitrophenyl)-thioureido]-2-phenyl acetamide (13.3 mg, 75.5%); from 2-Amino-N-(quinolin-6-yl)-2-phenylacetamide (10 mg, 0.036 mmoles) reacted with 2-methoxy-5-nitrophenylisothiocyanate (9.0 mg, 0.043 mmoles) in dichloromethane (1 ml) at 60° C. overnight.

Experiment 26:

C26 tert-butyl [1-(4-nitrophenylcarbamoyl)-1-phenyl-methyl]-carbamate

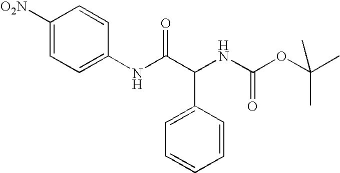

tert-butyl [1-(4-nitrophenylcarbamoyl)-1-phenyl-methyl]-carbamate (280 mg, 37.8%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being queched with 4-nitroaniline (331,2 mg, 2.4 mmoles).

D26 2-Amino-N-(4-nitrophenyl)-2-phenylacetamide

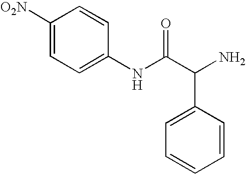

2-Amino-N-(4-nitrophenyl)-2-phenylacetamide (114.5 mg, 55.9%); from tert-butyl [1-(4-nitrophenylcarbamoyl)-1-phenyl-methyl]-carbamate (280 mg, 0.754 mmoles) reacted with formic acid (2 ml) at 60° C. for 0.5 hour.

E26.1 N-(4-nitrophenyl)-2-[3-(2-methoxy-5-nitrophenyl)-thioureido]-2-phenyl acetamide

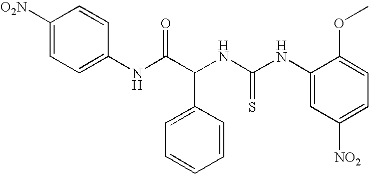

N-(4-nitrophenyl)-2-[3-(2-methox-5-nitrophenyl)-thioureido]-2-phenyl acetamide (23.4 mg, 91%); from 2-Amino-N-(4-nitrophenyl)-2-phenylacetamide (14.5 mg, 0.0535 mmoles) reacted with 2-methoxy-5-nitrophenylisothiocyanate (12.6 mg, 0.06 mmoles) in dichloromethane (1 ml) at 60° C. overnight.

E26.2 2-[3-(2-methoxy-5-nitro-phenyl)-ureido]-N-(4-nitrophenyl)-2-phenyl acetamide

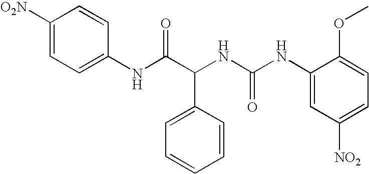

2-[3-(2-methoxy-5-nitro-phenyl)-ureido]-N-(4-nitrophenyl)-2-phenyl acetamide (25 mg, 97.6%); from 2-Amino-N-(4-nitrophenyl)-2-phenylacetamide (15 mg, 0.055 mmoles) reacted with 2-methoxy-5-nitrophenylisocyanate (15 mg, 0.077 mmoles) in dichloromethane (0.8 ml) at 60° C. overnight.

Experiment 27:

C27 tert-butyl [1-(4-trifluoromethoxyphenylcarbamoyl)-1-phenyl-methyl]-carbamate

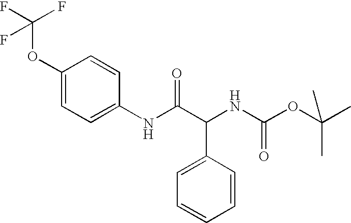

tert-butyl [1-(4-trifluoromethoxyphenylcarbamoyl)-1-phenyl-methyl]-carbamate (302 mg, 36.9%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2,2 mmoles) in THF (5 ml) at −40˜−50° C., followed by being queched with 4-trifluoromethoxyaniline (424.8 mg, 2.4 mmoles).

D27 2-Amino-N-(4-trifluoromethylphenyl)-2-phenylacetamide formic acid salt

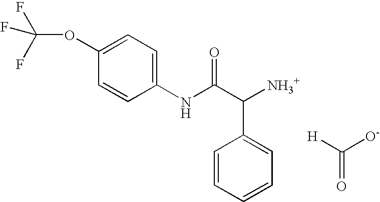

2-Amino-N-(4-trifluoromethyl phenyl)-2-phenylacetamide formic acid salt (221.5 mg, 91.1%); from tert-butyl [1-(4-trifluoromethoxyphenylcarbamoyl)-1-phenyl-methyl]-carbamate (280 mg, 0.682 mmoles) reacted with formic acid (2 ml) at 60° C. for 0.5 hour.

E27.1 N-(4-trifluoromethoxyphenyl)-2-[3-(2-methoxy-5-nitrophenyl)-thioureido]-2-phenyl acetamide

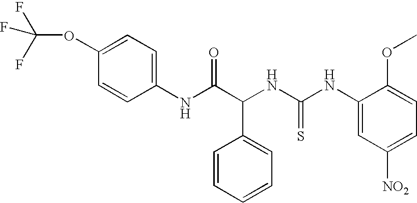

N-4-trifluoromethoxyphenyl)-2-[3-(2-methoxy-5-nitrophenyl)-thioureido]-2-phenyl acetamide (11.8 mg, 59.4%); from 2-Amino-N-(4-trifluoromethoxyyphenyl)-2-phenylacetamide (13.6 mg, 0.038 mmoles) reacted with 2-methoxy-5-nitrophenylisothiocyanate (12.6 mg, 0.06 mmoles) in dichloromethane (1 ml) at 60° C. overnight.

E27.2 2-[3-(2-methoxy-5-nitro-phenyl)-ureido]-N-(4-trifluoromethxyphenyl)-2-phenyl acetamide

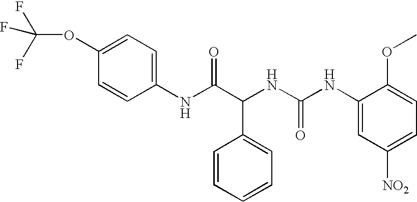

2-[3-(2-methoxy-5-nitro-phenyl)-ureido]-N-(4-trifluoromethxyphenyl)-2-phenyl acetamide (20.7 mg, 62.9%); from 2-Amino-N-(4-trifluoromethoxphenyl)-2-phenylacetamide (15 mg, 0.048 mmoles) reacted with 2-methoxy-5-nitrophenylisocyanate (15 mg, 0.077 mmoles) in dichloromethane (0.8 ml) at 60° C. overnight.

Experiment 28:

C28 tert-butyl [1-(4-methoxy phenylcarbamoyl)-1-phenyl-methyl]-carbamate

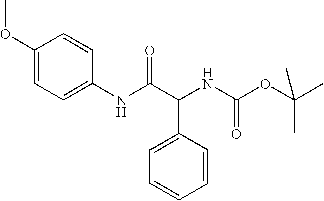

tert-butyl [1-(4-methoxyphenylcarbamoyl)-1-phenyl-methyl]-carbamate (546 mg, 76.9%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being queched with p-anisidine (295.6 mg, 2.4 mmoles).

D28 2-Amino-N-(4-methoxyphenyl)-2-phenylacetamide

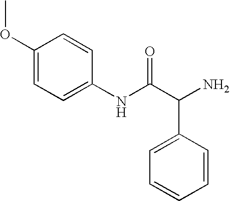

2-Amino-N-(4-methylphenyl)-2-phenylacetamide (313 mg, 87%); from tert-butyl [1-(4-methoxyphenylcarbamoyl)-1-phenyl-methyl]-carbamate (500 mg, 1.4 mmoles) reacted with formic acid (4 ml) at 60° C. for 0.5 hour.

E28.1 N-(4-methoxyphenyl)-2-[3-(2-methoxy-5-nitrophenyl)-thioureido]-2-phenyl acetamide

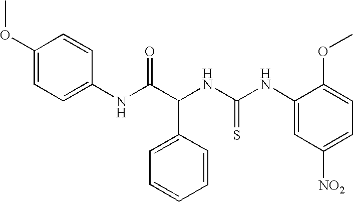

N-(4-methoxyphenyl)-2-[3-(2-methoxy-5-nitrophenyl)-thioureido]-2-phenyl acetamide (24.5 mg, 91%); from 2-Amino-N-(4-methoxyphenyl)-2-phenylacetamide (14.8 mg, 0.0578 mmoles) reacted with 2-methoxy-5-nitrophenylisothiocyanate (12.6 mg, 0.06 mmoles) in dichloromethane (1 ml) at 60° C. overnight.

E28.2 2-[3-(2-methoxy-5-nitro-phenyl)-ureido]-N-(4-methoxyphenyl)-2-phenyl acetamide

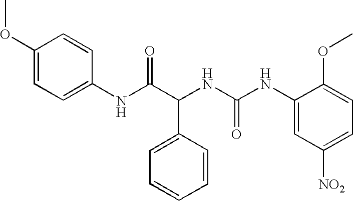

2-[3-(2-methoxy-5-nitro-phenyl)-ureido]-N-(4-methoxyphenyl)-2-phenyl acetamide (24.7 mg, 92.9%); from 2-Amino-N-(4-methoxyphenyl)-2-phenylacetamide (15 mg, 0.059 mmoles) reacted with 2-methoxy-5-nitrophenylisocyanate (15 mg, 0.077 mmoles) in dichloromethane (0.8 ml) at 60° C. overnight.

Experiment 29:

C29 tert-butyl [1-(indan-5-yl carbamoyl)-1-phenyl-methyl]-carbamate

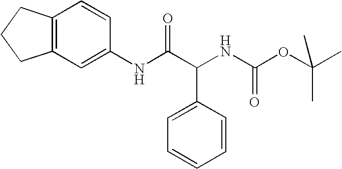

tert-butyl [1-(indan-5-yl carbamoyl)-1-phenyl-methyl]-carbamate (586 mg, 80.3%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being quenched with 5-aminoindane (424.8 mg, 2.4 mmoles).

D29 2-Amino-N-(indan-5-yl)-2-phenylacetamide

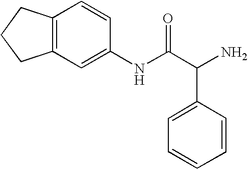

2-Amino-N-(indan-5-yl)-2-phenylacetamide (230 mg, 63.3%); tert-butyl [1-(indan-5-ylcarbamoyl)-1-phenyl-methyl]-carbamate (500mg, 1.364 mmoles) reacted with formic acid (4 ml) at 60° C. for 0.5 hour.

E29.1 N-(indan-5-yl)-2-[3-(2-methoxy-5-nitrophenyl)-thioureido]-2-phenyl acetamide

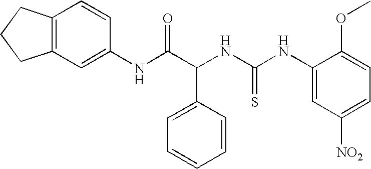

N-(indan-5-yl)-2-[3-(2-methoxy-5-nitrophenyl)-thioureido]-2-phenyl acetamide (22.4 mg, 84.5%); from 2-Amino-N-(indan-5-yl)-2-phenylacetamide (13.9 mg, 0.0556 mmoles) reacted with 2-methoxy-5-nitrophenylisothiocyanate (12.6 mg, 0.06 mmoles) in dichloromethane (1 ml) at 60° C. overnight.

E29.2 N-(indan-5-yl)-2-[3-(2-chloro-5-nitrophenyl)-thioureido]-2-phenyl acetamide

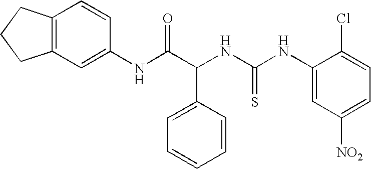

N-(indan-5-yl)-2-[3-(2-chloro-5-nitrophenyl)-thioureido]-2-phenyl acetamide (26.1 mg, 96.9%); from 2-Amino-N-(indan-5-yl)-2-phenylacetamide (15 mg, 0.056 mmoles) reacted with 2-chloro-5-nitrophenylthioisocyanate (15 mg, 0.07 mmoles) in dichloromethane (0.5 ml) at 60° C. overnight.

E29.3 N-(indan-5-yl)-2-[3-(2-methoxy-5-nitrophenyl)-ureido]-2-phenyl acetamide

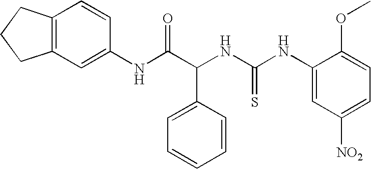

N-(indan-5-yl)-2-[3-(2-methoxy-5-nitrophenyl)-ureido]-2-phenyl acetamide (21 mg, 81.4%); from 2-Amino-N-(indan-5-yl)-2-phenylacetamide (15 mg, 0.056 mmoles) reacted with 2-methoxy-5-nitrophenylisocyanate (15 mg, 0.077 mmoles) in dichloromethane (0.8 ml) at 60° C. overnight.

C29* R-tert-butyl [1-(indan-5-yl-carbamoyl)-1-phenyl-methyl]-carbamate

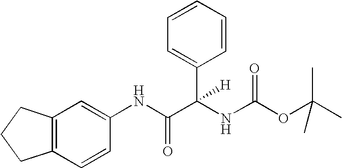

R-tert-butyl [1-(indan-5-yl carbamoyl)-1-phenyl-methyl]-carbamate (584.1 mg); from [(R-tert-butoxycarbonyl)amino](phenyl)acetic acid (600 mg, 2.39 mmoles) reacted with isobutyl chloroformate (339 μl, 2.62 mmoles) and N-methylmorpholine(288 μl, 2.62 mmoles) in THF (2 ml) at −78° C., followed by being quenched with 5-aminoindan(401 mg, 2.99 mmoles).

D29* R-2-Amino-N-(indan-5-yl)-2-phenylacetamide

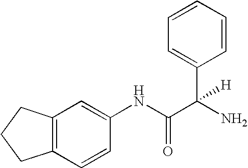

R-2-Amino-N-(indan-5-yl)-2-phenylacetamide (484.6 mg,); R-tert-butyl [1-(indan-5-ylcarbamoyl)-1-phenyl-methyl]-carbamate (584.1 mg) reacted with formic acid (2 ml) at 55° C. for 25 min.

E29.1* R-N-(indan-5-yl)-2-[3-(2-methoxy-5-nitrophenyl)-ureido]-2-phenyl acetamide

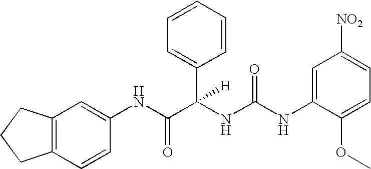

R-N-(indan-5-yl)-2-[3-(2-methoxy-5-nitrophenyl)-ureido]-2-phenyl acetamide (670.0 mg, 79.9%); from 2-Amino-N-(indan-5-yl)-2-phenylacetamide (484.6 mg, 1.8194 mmoles) reacted with 2-methoxy-5-nitrophenylisocyanate (459 mg, 2.365 mmoles) in dichloromethane (12 ml) at 60° C. for 2 hours.

Experiment 30:

C3 tert-butyl [1-(2-naphthylcarbamoyl)-1-phenyl-methyl]-carbamate

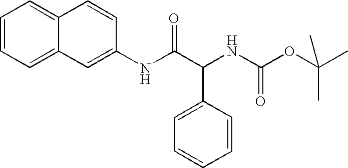

tert-butyl [1-(2-naphthylcarbamoyl)-1-phenyl-methyl]-carbamate (689 mg, 91.9%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being quenched with 2-aminonaphthalene (345.12 mg, 2.4 mmoles).

D30 2-Amino-N-(2-naphthyl)-2-phenylacetamide

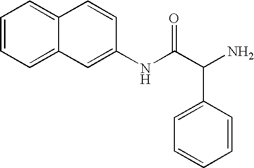

2-Amino-N-(2-naphthyl)-2-phenylacetamide (411 mg, 93.3%); from tert-butyl [1-(2-naphthylcarbamoyl)-1-phenyl-methyl]-carbamate (600 mg, 1.594 mmoles) reacted with formic acid (6 ml) at 60° C. for 0.5 hour.

E30.1 2-[3-(2-methoxy-5-nitro-phenyl)-thioureido]-N-(2-naphthyl)-2-phenyl acetamide

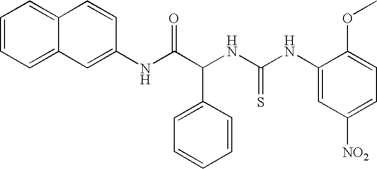

2-[3-(2-methoxy-5-nitro-phenyl)-thioureido]-N-(2-naphthyl)-2-phenyl acetamide (23.5 mg, 89.4%); from 2-Amino-N-(2-naphthyl)-2-phenylacetamide (15 mg, 0.054 mmoles) reacted with 2-methoxy-5-nitrophenylisothiocyanate (15 mg, 0.0714 mmoles) in dichloromethane (0.5 ml) at 60° C. overnight.

E30.2 2-[3-(2-methoxy-5-nitro-phenyl)-ureido]-N-(2-naphthyl)-2-phenyl acetamide

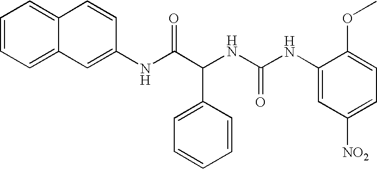

2-[3-(2-methoxy-5-nitro-phenyl)-ureido]- N-(2-naphthyl)-2-phenyl acetamide (16.5 mg, 64.9%); from 2-Amino-N-(2-naphthyl)-2-phenylacetamide (15 mg, 0.054 mmoles) reacted with 2-methoxy-5-nitrophenylisocyanate (15 mg, 0.077 mmoles) in dichloromethane (0.8 ml) at 60° C. overnight.

Experiment 31:

C31 tert-butyl [1-(3-trifluoromethylphenylcarbamoyl)-1-phenyl-methyl]-carbamate

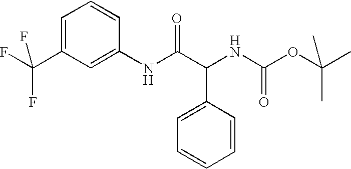

tert-butyl [1-(3-trifluoromethylphenylcarbamoyl)-1-phenyl-methyl]-carbamate (708 mg, 90.2%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being queched with 3-trifluoromethylaniline (386.4 mg, 2.4 mmoles).

D37 2-Amino-N-(3-trifluoromethylphenyl)-2-phenylacetamide

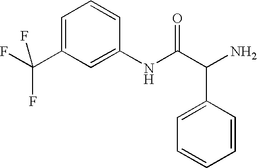

2-Amino-N-(3-trifluoromethylphenyl)-2-phenylacetamide (313 mg, 92%); from tert-butyl [1-(3-trifluoromethylphenylcarbamoyl)-1-phenyl-methyl]-carbamate (600 mg, 1.52 mmoles) reacted with formic acid (6 ml) at 60° C. for 0.5 hour.

D37* (R)-2-Amino-N-(3-trifluoromethylphenyl)-2-phenylacetamide

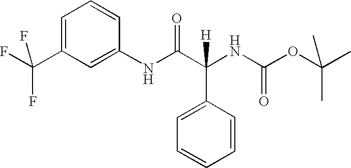

(R)-(−)-2-phenylglycine chloride hydrochloride (2.0 g, 0.97 mmoles) was mixed with tetrahydrofuran(10 ml) at −60° C., then 3-trifluoromethylaniline (3.374 g, 2.13 mmoles) was added. After being stirred for 1 hour, the reaction mixture was diluted with dichloromethane and washed by 1 M sodium hydroxide. The organic layer was dried with sodium sulfate, concentrated, triturated with hexanes to give (R)-2-Amino-N-(3-trifluoromethylphenyl)-2-phenylacetamide (2.3 g, yield: 80.5%)

E31.1 2-[3-(2-methoxy-5-nitro-phenyl)-thioureido]-N-(3-trifluoromethylphenyl)-2-phenyl acetamide

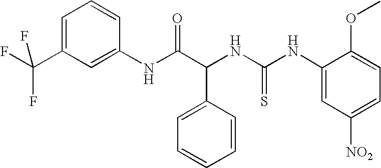

2-[3-(2-methoxy-5-nitro-phenyl)-thioureido]- N-(3-trifluoromethylphenyl)-2-phenyl acetamide (20.5 mg, 79.6%); from 2-Amino-N-(3-trifluoromethylphenyl)-2-phenylacetamide (15 mg, 0.051 mmoles) reacted with 2-methoxy-5-nitrophenylisothiocyanate (15 mg, 0.0714 mmoles) in dichloromethane (0.5 ml) at 60° C. overnight.

E31.2 2-[3-(2-methoxy-5-nitro-phenyl)-ureido]-N-(3-trifluoromethylphenyl)-2-phenyl acetamide

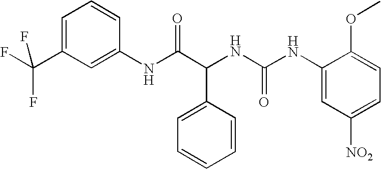

2-[3-(2-methoxy-5-nitro-phenyl)-ureido]-N-(3-trifluoromethylphenyl)-2-phenyl acetamide (25 mg, quantitative); from 2-Amino-N-(3-trifuromethylphenyl)-2-phenylacetamide (15 mg, 0.059 mmoles) reacted with 2-methoxy-5-nitrophenylisocyanate (15 mg, 0.077 mmoles) in dichloromethane (0.8 ml) at 60° C. overnight.

Experiment 32:

C32 tert-butyl [1-(3,4-dimethylphenylcarbamoyl)-1-phenyl-methyl]-carbamate

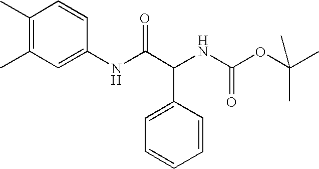

tert-butyl [1-(3,4-dimethylphenylcarbamoyl)-1-phenyl-methyl]-carbamate (666 mg, 94.4%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being quenched with 3,4-dinethylaniline (290.18 mg, 2.4 mmoles).

D32 2-Amino-N-(3,4-dimethylphenyl)-2-phenylacetamide

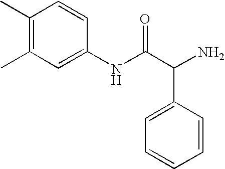

2-Amino-N-(3,4-dimethylphenyl)-2-phenylacetamide (416 mg, 96.6%); from tert-butyl [1-(3,4-dimethylphenylcarbamoyl)-1-phenyl-methyl]-carbamate (600 mg, 1.93 mmoles) reacted with formic acid (6 ml) at 60° C. for 0.5 hour.

E32.1 2-[3-(2-chloro-5-nitro-phenyl)-thioureido]-N-(3,4-dimethylphenyl)-2-phenyl acetamide

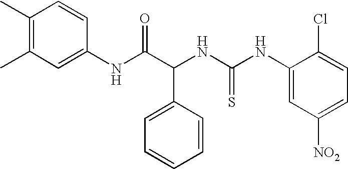

2-[3-(2-chloro-5-nitro-phenyl)-thioureido]- N-(3,4-dimethylphenyl)-2-phenyl acetamide (19.1 mg, 69%); from 2-Amino-N-(3,4-dimethylphenyl)-2-phenylacetamide (15 mg, 0.059 mmoles) reacted with 2-chloro-5-nitrophenylisothiocyanate (15 mg, 0.07 mmoles) in dichloromethane (0.5 ml) at 60° C. overnight.

E32.2 2-[3-(2-methoxy-5-nitro-phenyl)-thioureido]-N-(3,4-dimethylphenyl)-2-phenyl acetamide

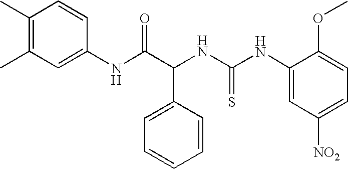

2-[3-(2-methoxy-5-nitro-phenyl)-thioureido]-N-(3,4-dimethylphenyl)-2-phenyl acetamide (19.5 mg, 71.1%); from 2-Amino-N-(3,4-dimethylphenyl)-2-phenylacetamide (15 mg, 0.059 mmoles) reacted with 2-methoxy-5-nitrophenylisothiocyanate (15 mg, 0.0714 mmoles) in dichloromethane (0.5 ml) at 60° C. overnight.

E32.3 2-[3-(2-fluoro-5-nitro-phenyl)-ureido]-N-(3,4-dimethylphenyl)-2-phenyl acetamide

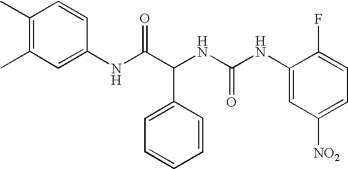

2-[3-(2-fluoro-5-nitro-phenyl)-ureido]-N-(3,4-dimethylphenyl)-2-phenyl acetamide (13.4 mg, 87%); from 2-Amino-N-(3,4-dimethylphenyl)-2-phenylacetamide (9 mg, 0.0354 mmoles) reacted with 2-fluoro-5-nitrophenylisocyanate (9 mg, 0.0494 mmoles) in dichloromethane (1 ml) at 60° C. overnight.

E32.4 2-[3-( 2-methoxy-5-nitro-phenyl)-ureido]-N-(3,4-dimethylphenyl)-2-phenyl acetamide

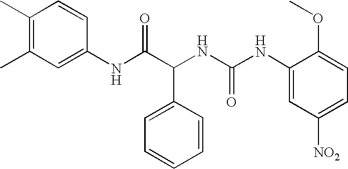

2-[3-(2-methoxy-5-nitro-phenyl)-ureido]-N-(3,4-dimethylphenyl)-2-phenyl acetamide (22.1 mg, 83.5%); from 2-Amino-N-(3,4-dimethylphenyl)-2-phenylacetamide (15 mg, 0.059 mmoles) reacted with 2-methoxy-5-nitrophenylisocyanate (15 mg, 0.077 mmoles) in dichloromethane (8 ml) at 60° C. overnight.

E32.5 (R)-2-[3-(2-methoxy-5-nitro-phenyl)-ureido]-N-(3,4-dimethylphenyl)-2-phenyl acetamide

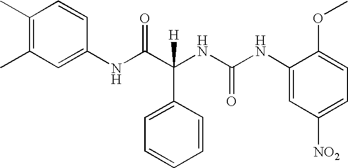

(R)-2-[3-(2-methoxy-5-nitro-phenyl)-ureido]-N-(3,4-dimethylphenyl)-2-phenyl acetamide (129 mg, 83.5%); from (R)-2-Amino-N-(3,4-dimethylphenyl)-2-phenylacetamide (84 mg, 0.237 mmoles) reacted with 2-methoxy-5-nitrophenylisocyanate (110 mg, 0.567 mmoles) in dichloromethane (5 ml) at 60° C. overnight.

E32.6 2-[3-(2-(N,N-dimethylaminoethoxy-5-nitro-phenyl)-ureido]-N-3,4-dimethylphenyl)-2-phenyl acetamide

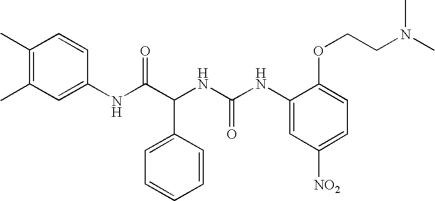

2-(N,N-dimethylaminoethoxy)-5-nitroaniline (25 mg, 0.11 mmoles) was mixed with triphosgene (32 mg) in dichloromethane at ambient temperature for 0.5 hour. Then 2-Amino-N-(3,4-dimethylphenyl)-2-phenylacetamide (40 mg, 0.157 mmoles) was added and heated to 60° C. for 1 hour. The product was diluted with dichloromethane and purified by column chromatography with 1.5% methanol (2MNH₃) in dichloromethane to give 2-[3-(2-(N,N-dimethylaminoethoxy-5-nitro-phenyl)-ureido]-N-(3,4-dimethylphenyl)-2-phenyl acetamide (3.2 mg, 5.7%)

E32.7 2-[3-(2-methoxy-5-nitro-phenyl)-cyano-quanadine]-N-(3,4-dimethylphenyl)-2-phenyl acetamide

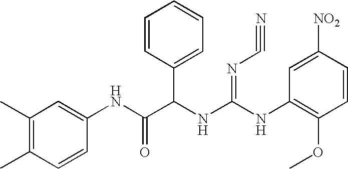

2-[3-(2-methoxy-5-nitro-phenyl)-cyano-guanadine]-N-(3,4-dimethylphenyl)-2-phenyl acetamide (21.2 mg, 11%) was isolated as a white solid. The title compound was made by reaction of Sodium t-butoxide (0.492 mmol) with a solution of Cyanamide (20 mg, 0.492 mmol) in DMF (3 ml) at room temperature for 20 min followed by the addition of 2-methoxy-5-nitrophenylisothiocyanate (50 mg, 0.492 mmol). The solution was then cooled to 0° C. and triethylamine (0.096 ml, 0.688 mmol) was added. Next the starting material 2-Amino-N-(3,4-dimethylphenyl)-2-phenylacetamide (100 mg, 0.393 mmol) was added and finally HgCl₂ (112.4 mg, 0.443 mmol). The reaction mixture was stirred at 0° C. for 2 hours. The final product was isolated from the reaction mixture by dilution in ethylacetate followed by washing with water and then with brine. The organic extracts were dried over Na₂SO₄ and concentrated to dryness. The product was purified by flash chromatography on Silica gel with a gradient elution of 10% to 40% Ethylacetate in Hexane.

Experiment 33:

C33* (R)-tert-butyl [1-(3,4-dimethylphenylcarbamoyl)-1-phenyl-methyl]-carbamate

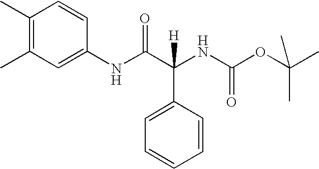

(R)-tert-butyl [1-(3,4-dimethylphenylcarbamoyl)-1-phenyl-methyl]-carbamate (613 mg, 86.9%); from (R)-[(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −78° C., followed by being quenched with 3,4-dimethylaniline (290.18 mg, 2.4 mmoles).

D33* (R)-2-Amino-N-(3,4-dimethylphenyl)-2-phenylacetamide

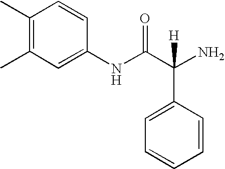

(R)-2-Amino-N-(3,4-dimethylphenyl)-2-phenylacetamide (84 mg, 97.4%); from (R)-tert-butyl [1-(3,4-dimethylphenylcarbamoyl)-1-phenyl-methyl]-carbamate (120 mg, 0.039 mmoles) reacted with formic acid (0.5 ml) at 60° C. for 0.5 hour.

E33.1 * (R)-2-[3-(2-methoxy-5-nitro-phenyl)-thioureido]-N-(3,4-dimethylphenyl)-2-phenyl acetamide

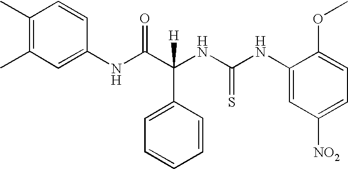

(R)-2-[3-(2-methoxy-5-nitro-phenyl)-thioureido]- N-(3,4-dimethylphenyl)-2-phenyl acetamide (456 mg, 83%); from (R)-2-Amino-N-(3,4-dimethylphenyl)-2-phenylacetamide (300 mg, 1.18 mmoles) reacted with 2-methoxy-5-nitrophenylisothiocyanate (300 mg, 1.43 mmoles) in dichloromethane (0.5 ml) at 60° C. overnight

Experiment 34:

C34 tert-butyl [1-(3,5-dimethylphenylcarbamoyl)-1-phenyl-methyl]-carbamate

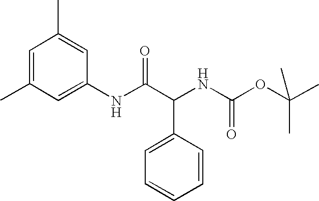

tert-butyl [1-(3,5-dimethylphenylcarbamoyl)-1-phenyl-methyl]-carbamate (620 mg, 87.8%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being queched with 3,5-dimethylaniline (290.8 mg, 2.4 mmoles).

D34 2-Amino-N-(3,5-dimethylphenyl)-2-phenylacetamide

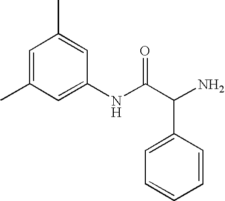

2-Amino-N-(3,5-dimethylphenyl)-2-phenylacetamide (365 mg, 83.5%); from tert-butyl [1-(3,5-dimethylphenylcarbamoyl)-1-phenyl-methyl]-carbamate (609 mg, 1.718 mmoles) reacted with formic acid (5 ml) at 60° C. for 0.5 hour.

E34.1 2-[3-(2-chloro-5-nitro-phenyl)-thioureido]-N-(3,5-dimethylphenyl)-2-phenyl acetamide

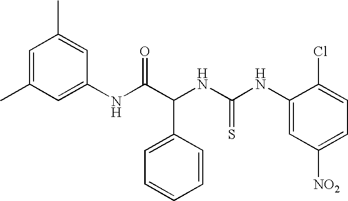

2-[3-(2-chloro-5-nitro-phenyl)-thioureido]-N-(3,5-dimethylphenyl)-2-phenyl acetamide (24.3 mg, 87.8%); from 2-Amino-N-(3,5-dimethyphenyl)-2-phenylacetamide (15 mg, 0.059 mmoles) reacted with 2-chloro-5-nitrophenylisothiocyanate (15 mg, 0.07 mmoles) in dichloromethane (0.5 ml) at 60° C. overnight.

E34.2 2-[3-(2-methoxy-5-nitro-phenyl)-thioureido]-N-(3,5-dimethylphenyl)-2-phenyl acetamide

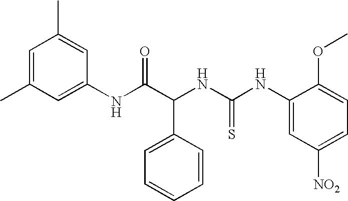

2-[3-(2-methoxy-5-nitro-phenyl)-thioureido]-N-(3,5-dimethylphenyl)-2-phenyl acetamide (27.4 mg, quantitative); from 2-Amino-N-(3,5-dimethylphenyl)-2-phenylacetamide (15 mg, 0.059 mmoles) reacted with 2-methoxy-5-nitrophenylisothiocyanate (15 mg, 0.0714 mmoles) in dichloromethane (0.5 ml) at 60° C. overnight.

E34.3 2-[3-(2-methoxy-5-nitro-phenyl)-ureido]-N-(3,5-dimethylphenyl)-2-phenyl acetamide

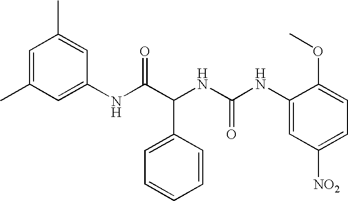

2-[3-(2-methoxy-5-nitro-phenyl)-ureido]-N-(3,5-dimethylphenyl)-2-phenyl acetamide (25.6 mg, 96.7%); from 2-Amino-N-(3,5-dimethylphenyl)-2-phenylacetamide (15 mg, 0.059 mmoles) reacted with 2-methoxy-5-nitrophenylisocyanate (15 mg, 0.077 mmoles) in dichloromethane (0.8 ml) at 60° C. overnight.

Experiment 35:

C35 tert-butyl [1-(4-vinylphenylcarbamoyl)-1-phenyl-methyl]-carbamate

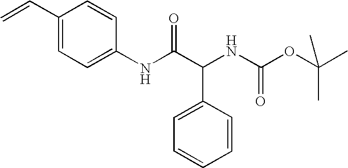

tert-butyl [1-(4-vinylphenylcarbamoyl)-1-phenyl-methyl]-carbamate (610 mg, 86.4%); from [(tert-butoxycarbonyl)amino](phenyl)acetic acid (500 mg, 2 mmoles) reacted with isobutyl chloroformate (235 μl, 2.2 mmoles) and N-methylmorpholine(241 μl, 2.2 mmoles) in THF (5 ml) at −40˜−50° C., followed by being quenched with 4-vinylaniline (290.8 mg, 2.4 mmoles).

D35 2-Amino-N-(4-vinylphenyl)-2-phenylacetamide

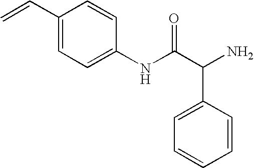

2-Amino-N-(4-vinylphenyl)-2-phenylacetamide (188 mg, 44.7%); from tert-butyl [1 -(4-vinylphenylcarbamoyl)-1-phenyl-methyl]-carbamate (587 mg, 1.66 mmoles) reacted with formic acid (5 ml) at 60° C. for 0.5 hour.

E35.1 2-[3-(2-chloro-5-nitro-phenyl)-thioureido]-N-(4-vinylphenyl)-2-phenyl acetamide

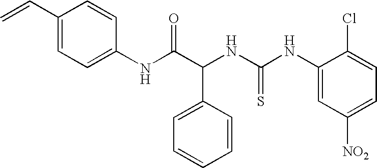

2-[3-(2-chloro-5-nitro-phenyl)-thioureido]-N-(4-vinylphenyl)-2-phenyl acetamide (15.2 mg, 55.1%); from 2-Amino-N-(4-vinylphenyl)-2-phenylacetamide (15 mg, 0.059 mmoles) reacted with 2-chloro-5-nitrophenylisothiocyanate (15 mg, 0.07 mmoles) in dichloromethane (0.5 ml) at 60° C. overnight.

E35.2 2-[3-(2-methyoxy-5-nitro-phenyl)-thioureido]-N-(4-vinylphenyl)-2-phenyl acetamide

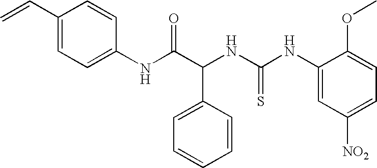

2-[3-(2-methoxy-5-nitro-phenyl)-thioureido]-N-(4-vinylphenyl)-2-phenyl acetamide (21.7 mg, 79.5%); from 2-Amino-N-(4-vinylphenyl)-2-phenylacetamide (15 mg, 0.059 mmoles) reacted with 2-methoxy-5-nitrophenylisothiocyanate (15 mg, 0.0714 mmoles) in dichloromethane (0.5 ml) at 60° C. overnight.

E35.3 2-[3-(2-methoxy-5-nitro-phenyl)-ureido]-N-(4-vinylphenyl)-2-phenyl acetamide

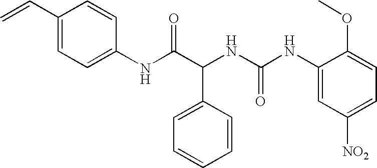

2-[3-(2-methoxy-5-nitro-phenyl)-ureido]-N-(4-vinylphenyl)-2-phenyl acetamide (22.6 mg, 85.4%); from 2-Amino-N-(4-vinylphenyl)-2-phenylacetamide (15 mg, 0.059 mmoles) reacted with 2-methoxy-5-nitrophenylisocyanate (15 mg, 0.077 mmoles) in dichloromethane (0.8 ml) at 60° C. overnight.

Experiment 36:

C36 tert-butyl-[1-(6-m thylpyridin-3-ylcarbamoyl)-1-phenyl-methylcarbamate

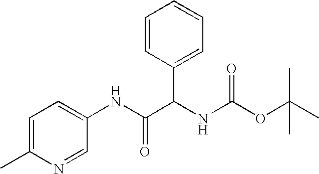

tert-butyl-[1-(6-methylpyridin-3-ylcarbamoyl)-1-phenyl-methylcarbamate (235 mg, 74.4%); from [(tert-butoxycarbonyl)amino](phenyl)acetic acid (279 mg, 1.11 mmoles) reacted with isobutyl chloroformate (130 μl, 1.22 mmoles) and N-methylmorpholine (133 μl, 1.22 mmoles) in THF (2.5 ml) at −40˜−50° C., followed by being queched 3-amino-6-methylpyridine (100 mg, 0.925 mmoles).

D36 2-Amino-N-(6-methylpyridin-3-yl)-2-phenylacetamide

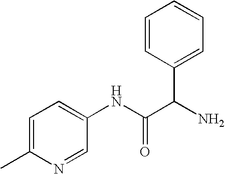

2-Amino-N-(6-methylpyridin-3-yl)-2-phenylacetamide (87.3 mg, 52.9%); from tert-butyl-[1-(6-methylpyridin-3-ylcarbamoyl)-1-phenyl-methylcarbamate (235 mg, 0.688 mmoles) reacted with formic acid (2.5 ml) at 60° C. for 0.5 hour.

E36.1 2-[3-(2-methoxy-5-nitro-phenyl)-ureido]-N-(6-methylpyridin-3-yl)-2-phenyl acetamide

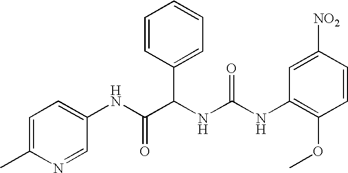

2-[3-(2-methoxy-5-nitro-phenyl)-ureido]-N-(6-methylpyridin-3-yl)-2-phenyl acetamide (17.5 mg, 98.2%); from 2-Amino-N-(6-methylpyridin-3-yl)-2-93 phenylacetamide (10 mg, 0.041 mmoles) reacted with 2-methoxy-5-nitrophenylisocyanate (10 mg, 0.052 mmoles) in dichloromethane (0.5 ml) at 60° C. overnight.

Experiment 37

C37 tert-butyl-[1-(6-trifluoromethylpyridin-3-ylcarbamoyl)-1-phenyl-methylcarbamate

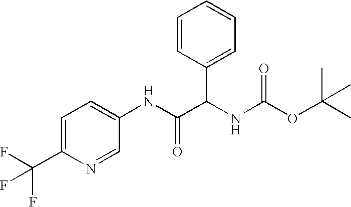

tert-butyl-[1-(6-trifluoromethylpyridin-3-ylcarbamoyl)-1-phenyl-methylcarbamate (477 mg, 55.8%); from [(tert-butoxycarbonyl)amino](phenyl)acetic acid (500 mg, 2 mmoles) reacted with isobutyl chloroformate (235 μl, 2.2 mmoles) and N-methylmorpholine (241 μl, 2.2 mmoles) in THF (5 ml) at −40˜−50° C., followed by being quenched 3-amino-6-trifluoromethylpyridine (350 mg, 2.16 mmoles).

D37 2-Amino-N-(6-trifluoromethylpyridin-3-yl)-2-phenylacetamide

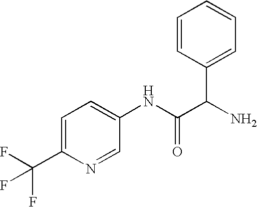

2-Amino-N-(6-trifluoromethylpyridin-3-yl)-2-phenylacetamide (317 mg, 89%); from tert-butyl-[1-(6-trifluoromethylpyridin-3-ylcarbamoyl)-1-phenyl-methylcarbamate (477 mg, 1.397 mmoles) reacted with formic acid (5 ml) at 60° C. for 0.5 hour.

E37.1 2-[3-(2-methoxy-5-nitro-phenyl)-ureido]-N-(6-trifluoromethylpyridin-3-yl)-2-phenyl acetamide

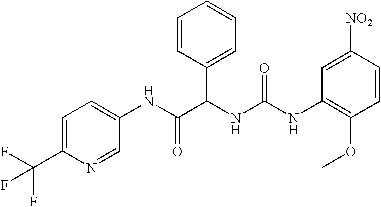

2-[3-(2-methoxy-5-nitro-phenyl)-ureido]-N-(6-trifluoromethylpyridin-3-yl)-2-phenyl acetamide (16.3 mg, 97.9%); from 2-Amino-N-(6-trifluoromethylpyridin-3-yl)-2-phenylacetamide (10 mg, 0.034 mmoles) reacted with 2-methoxy-5-nitrophenylisocyanate (10 mg, 0.052 mmoles) in dichloromethane (0.5 ml) at 60° C. overnight.

E37.2 2-[3-(2-methoxy-5-nitro-phenyl)-thioureido]-N-(6-trifluoromethylpyridin-3-yl)-2-phenyl acetamide

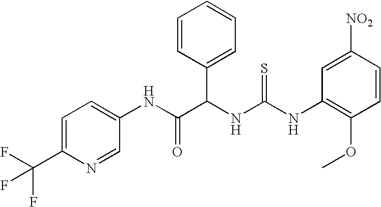

2-[3-(2-methoxy-5-nitro-phenyl)-thioureido]-N-(6-trifluoromethylpyridin-3-yl)-2-phenyl acetamide (12.6 mg, 73.3%); from 2-Amino-N-(6-trifluoromethylpyridin-3-yl)-2-phenylacetamide (10 mg, 0.034 mmoles) reacted with 2-methoxy-5-nitrophenylisothiocyanate (10 mg, 0.048 mmoles) in dichloromethane (0.5 ml) at 60° C. overnight.

Experiment 38:

E38.1 N-(4-bromophenyl)-2-[3-(2-methoxy-5-nitrophenyl)-thioureido]-2-phenyl acetamide

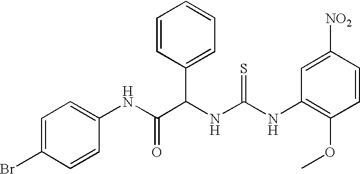

N-(4-bromophenyl)-2-[3-(2-nitrophenyl)-thioureido]-2-phenyl acetamide (23.5 mg, 95.9%); from 2-Amino-N-(4-bromoyphenyl)-2-phenylacetamide (14.5 mg, 0.0475 mmoles) reacted with 2-methoxy-5-nitrophenylisothiocyanate (12.6 mg, 0.06 mmoles) in dichloromethane (1 ml) at 60° C. overnight.

E38.2 2-[3-(2-methoxy-5-nitro-phenyl)-ureido]-N-(4-bromophenyl)-2-phenyl acetamide

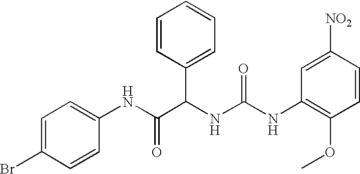

2-[3-(2-methoxy-5-nitro-phenyl)-ureido]-N-(4-bromophenyl)-2-phenyl acetamide (19.7 mg, 80.5%); from 2-Amino-N-(4-bromophenyl)-2-phenylacetamide (15 mg, 0.049 mmoles) reacted with 2-methoxy-5-nitrophenylisocyanate (15 mg, 0.077 mmoles) in dichloromethane (0.8 ml) at 60° C. overnight.

Experiment 39:

C39* R-tert-butyl[1-(4-methylphenylcarbamoyl)-1-cyclohexylmethyl]-carbamate

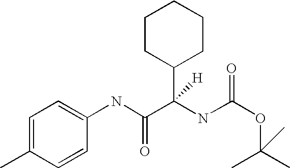

R-tert-butyl[1-(4-methylphenylcarbamoyl)-1-cyclohexylmethyl]-carbamate (96.9 mg) was isolated as a white solid from N-tert-butoxycarbonyl D-cyclohexyl glycine (250 mg, 1.1658 mmol), N-methylmorpholine (0.140 ml, 1.2823 mmol), isobutylchloroformate (0.166 ml, 1.2823 mmol), p-toluidine (0.149 mg, 1.398 mmol), and N-methylmorpholine (0.152 ml, 1.398 mmol).

D39* R-1-Amino-N-(4-methylphenyl)cyclohexane carboxamide

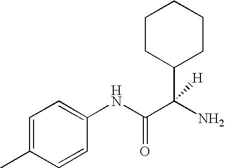

R-1 -Amino-N-(4-methylphenyl)cyclohexane carboxamide (71.1 mg), was isolated as a white solid from, R-tert-butyl[1-(4-methylphenylcarbamoyl)-1-cyclohexylmethyl]-carbamate (96.0 mg) stirred with formic acid (2 ml) 50° C. for 2 hours.

E39* R-[1-(2-methoxy-4-nitrophenyl)-thiouriedo]-N-(4-methylphenyl)-cyclohexane carboxamide

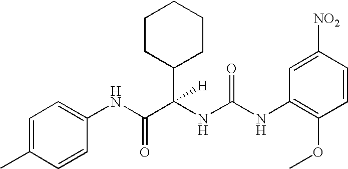

R-[1-(2-methoxy-4-nitrophenyl)-thiouriedo]-N-(4-methylphenyl)-cyclohexane carboxamide (22.9 mg) was isolated as a white solid from R-1-Amino-N-(4-methylphenyl)cyclohexane carboxamide (35 mg, 0.144 mmol) and 2-methoxy-5-nitrophenylisothiocyanate (39.35 mg, 0.1873 mmol).

Experiment 40:

C40 Tert-butyl-[1-(2-cyclohexyl carbamoyl) -1-(3,4-dimethylphenyl)] carbamate

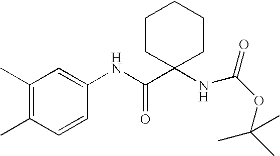

Tert-butyl-[1-(2-cyclohexyl carbamoyl)-1-(3,4-dimethylphenyl)] carbamate (265.3 mg) was isolated as a white solid from the corresponding non-natural amino acid (300 mg, 1.233 mmol) and N-methylmorpholine (0.15 ml, 1.356 mmol), Isobutylchloroformate (0.175 ml, 1.356 mmol), 3,4-dimethylaniline (179 mg, 1.479 mmol), and N-methylmorpholine (0.16 ml, 1.479 mmol).

D40 2-Amino-N-(3,4-dimethylphenyl)-2-cyclohexyl carboxamide

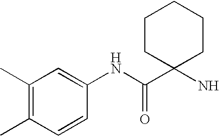

2-Amino-N-(3,4-dimethylphenyl)-2-cyclohexyl carboxamide was isolated as a white solid from Tert-butyl-[1-(2-cyclohexyl carbamoyl)-1-(3,4-dimethylphenyl)] carbamate stirred with formic acid (3 ml) at 50° C. for 2 hours.

E40 [1-(2-methoxy-5-nitrophenyl)-thioureido]-N-(3,4-dimethylphenyl)]-cyclohexane carboxamide

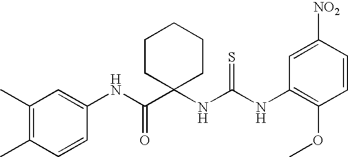

[1-(2-methoxy-5-nitrophenyl)-thioureido N-(3,4-dimethylphenyl)]-cyclohexane carboxamide (110 mg) was isolated from 2-Amino-N-(3,4-dimethylphenyl)-2-cyclohexyl carboxamide (50 mg 0.2029 mmol) and 2-methoxy-5-nitrophenylisothiocyanate (55.4 mg, 0.2637 mmol).

Experiment 41

C41 tert-butyl[1-(3,4-dimethylphenylcarbamoyl)-1-(3-thienyl)-methyl]-carbamate

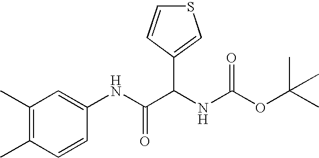

tert-butyl[1-(3,4-dimethylphenylcarbamoyl)-1-(3-thienyl)-methyl]-carbamate (Crude product used in next reaction); from [(tert-butoxycarbonyl)amino](thienyl)acetic acid (300 mg, 1.165 mmoles) reacted with isobutyl chloroformate (166 μl, 1.282 mmoles) and N-methylmorpholine(0.141 ml, 1.282 mmoles)-2-methyl-4-chloroaniline (169.1 mg, 1.19 mmol) and N-methylmorpholine (0.154 mL, 1.398 mmol).

D40 2-Amino-N-(3,4-dimethylphenyl)-2-thienylacetamide

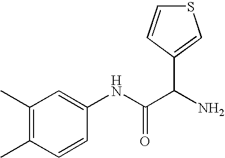

2-Amino-N-(3,4-dimethylphenyl)-2-(3-thienyl)-acetamide (96 mg); from tert-butyl[1-(3,4-dimethylphenylcarbamoyl)-1-(3-thienyl)methyl]-carbamate (Crude product from previous reaction)) reacted with formic acid (3 ml) at 60° C. for 0.5 hour.

E41.1 2-[3-(2-methoxy-5-nitro-phenyl)-ureido]-N-(3.5-dimethylphenyl)-2-(3-thienyl)-acetamide

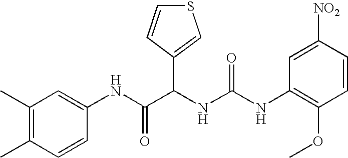

2-[3-(2-methoxy-5-nitro-phenyl)-ureido]-N-(3,5-dimethylphenyl)-2-(3-thienyl)-acetamide (73.54 mg, 98%) was isolated as an off white solid from 2-Amino-N-(3,4-dimethylphenyl)-2-(3-thienyl)-acetamide (45 mg, 0.165 mmol) and 2-methoxy-5-nitrophenylisocyanate (41.7 mg, 0.2147 mmol).

E41.2 2-[3-(2-methoxy-5-nitro-phenyl)-thioureido]-N-(3,5-dimethylphenyl)-2-(3-thienyl) acetamide

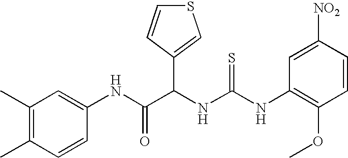

2-[3-(2-methoxy-5-nitro-phenyl)-thioureido]-N-(3,5-dimethylphenyl)-2-(-3-thienyl)-acetamide (71.95 mg, 92%) was isolated as a pale yellow solid from 2-Amino-N-(3,4-dimethylphenyl)-2-(3-thienyl)-acetamide (45 mg, 0.165 mmol) and 2-methoxy-5-nitrophenylisothiocyanate (45.13 mg, 0.2147 mmol).

Experiment 42

C42 (+/−)-Boc-amino-3-thienyl-glycine indan-3-ylamide

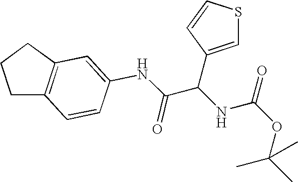

Isobutyl chloroformate (0.7 mL) was added to (+/−)-Boc-amino-3-thienyl-glycine (1.505 g, 5.85 mmol) and N-methylmorpholine (0.7 mL) in THF (15 mL) and the reaction mixture was stirred at −78° C. for 1 h. A mixture of 3-aminoindan (950 mg, 7.13 mmol) and N-methylmorpholine (0.7 mL) in THF (7 mL) was added and the resulting mixture was stirred for 2 h, slowly warming to RT. The mixture was partitioned between dichloromethane (200 mL) and water (50 mL). The organic layer was washed sequentially with water (50 mL), HCl (1 M, 50 mL), and brine (50 mL) and dried over sodium sulfate. Filtration through a small plug of silica gel followed by evaporation to remove the solvent yielded a solid, which was triturated with hexane to provide a product (1.82 g, 84%) sufficiently pure for the next step.

D42 (+/−)-3-thienyl-glycine indan-3-ylamide

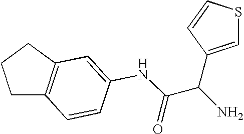

A mixture of (+/−)-Boc-amino-3-thienyl-glycine indan-3-ylamide (1.82 g, 4.89 mmol) and formic acid (96%, 20 mL) under Ar(g) washeated at 60° C. for 35 min. After cooling to RT, the formic acid was removed in vacuo. The crude product was taken up in dichloromethane (200 mL) and washed sequentially with NaOH (1 M, 100 mL) and brine (100 mL), dried over sodium sulfate, and the solvent was removed in vacuo. The oily residue was solidified by pumping, and then triturated using hexane to provide a product (1.19 g, 89%) sufficiently pure for the next step.

E42.1 N-(indan-5-yl)-2-[-3-(2-methoxy-5-nitrophenyl)-uerido-2-(3-thienyl-acetamide

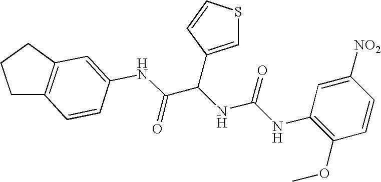

From the amine (351.7 mg, 1.29 mmol) and 2-methoxy-5-nitrophenylisocyanate (318 mg, 1.64 mmol) in dichloromethane (10 mL) at 60° C. for 3h. Collected precipitate and rinsed using dichloromethane (5×5 mL). Product was a yellow solid (605.6 mg, 100% yield, %, HRMS-FAB⁺ for C₂₃H₂₂N₄O₅S: calculated MH⁺:483.13270; found:483.11331).

E42.2 N-(indan-5-yl)-2-[-3-(2-methoxy-5-nitrophenyl)-thiouerido-2-(3-thienyl-acetamide

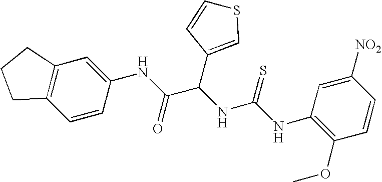

From the amine (351.0 mg, 1.29 mmol) and 2-methoxy-5-nitrophenylisothiocyanate (345 mg, 1.64 mmol) in dichloromethane (10 mL) at 60° C. for 3.5 h. Collected precipitate formed by cooling in ice and rinsed using dichloromethane (5×5 mL). Product was an off-white solid (577.5 mg, 93% yield, %, HRMS-FAB⁺ for C₂₃H₂₂N₄O₄S₂: calculated MH⁺:467.13892; found:467.13985).

Experiment 43

Assay of Transport via GlyT-1

This example illustrates a method for the measurement of glycine uptake by transfected cultured cells.

Cells stably transfected with GlyT-1 C (see Kim, et al., Molecular Pharmacology, 45, 1994:608-617) were washed twice with HEPES buffered saline (HBS). The cells were then incubated for 10 minutes at 37° C. with either (a) no potential competitor, (b) 10 mM non-radioactive glycine or (c) a concentration of a candidate drug. A range of concentrations of the candidate drug was used to generate data for calculating the concentration resulting in 50% of the effect (e.g., the IC₅₀s, which are the concentrations of drug inhibiting glycine uptake by 50%). A solution was then added containing [³H]glycine at a final concentration of 50 nM (17.5 Ci/mmol). The cells were then incubated with gentle shaking for another 30 minutes at 37° C., after which the reaction mixture was aspirated and washed three times with ice-cold HBS. The cells were lysed with scintillant and allowed to equilibrate. The radioactivity in the cells was determined using a scintillation counter. Data was compared between the same cells contacted or not contacted by a candidate agent, depending on the assay being conducted.

The compounds of the present invention were active as GlyT-1 inhibitors. The following table provides examples of the glycine uptake IC50 values for representative compounds of the invention.

| Experiment Number | GlyT1 uptake IC50 (nM) |
|---|---|
| E42.2 | 131.4488 |
| E32.2 | 333.9854 |
| E32.5 | 25.9085 |
| E33.1* | 96.0385 |
| E42.1 | 113.714 |
| E29.1* | 71.9265 |
| E4.1 | 67.3383 |
| E31.2 | 202.0483 |

### Example 43

Assay of Binding to NMDA Receptors-associated Glycine Binding Site

This example illustrates a method used to measure the interaction of compounds to the glycine site on the NMDA receptor. In this assay a known NMDA glycine site binding agent, (tritiated-MDL 105519, available from Amersham), is used to bind to rat hippocampal tissue. The test compound is then introduced and allowed to displace the hot ligand. Binding of the test compound will displace the hot ligand and result in reduced radioactivity, which can be quantified. Compounds are generally tested at two concentrations if inhibition is observed the compounds are retested at several concentrations to generate a dose response curve from which an IC50 may be determined.

The test compounds are prepared for the assay by diluting with 50 mM Tris Acetate buffer. Rat hippocampal membrane aliquots used in the assay are washed twice with cold 10 mM Tris Acetate buffer and subjected to ultracentrifugation at 20,000 rpm for 15 minutes, and rehomogenization between washes. The final pellets are then resuspended in 50 mM Tris Acetate buffer to provide the membranes at a concentration appropriate to the assay. Non-specific binding is defined in the presence of 1 mM glycine. Total binding is defined by the presence of Tris acetate buffer only.

The reaction mixture is prepared by combining 75 μg of homogenized hippocampal membrane preparation with [3H]-MDL 105519 to a final concentration of 5 nM and glycine or test compound as a solution in Tris Acetate Buffer. The reaction is shaken while incubating at room temp for 30 minutes. The plates are then harvested onto GFC filters using a 48 w Brandell Harvestor. The GFC filters are pre-treated for at least 30 minutes with a solution of 0.5% BSA made in distilled water to reduce non-specific binding of the hot ligand to the filter. The plate wells are washed with 4-5 volumes of cold 50 mM Tris Acetate buffer. The filters are then transferred to scintillation vials and 2 mls of scintillant is added to each vial. The vials are allowed to sit overnight before being counted in a Beckman β-counter. The data is analyzed using Prism software.

The compounds of the present invention show no significant binding to the NMDA receptor-associated glycine binding site.

### Example 44

Glycine Receptor Binding Assay

This example illustrates an assay used to measure cross reactivity of the compounds with the Glycine receptor. In this assay the known glycine receptor binding agent, [3H]-Strychnine is used to bind to rat spinal cord tissue. The test compound is then introduced and allowed to displace the hot ligand. Binding of the test compound will displace the hot ligand and result in reduced radioactivity, which can be quantified. Compounds are generally tested at two concentrations, if inhibition is observed the compounds are retested at several concentrations to generate a dose response curve from which an IC50 may be determined.

The test compounds are prepared for the assay by diluting in potassium phosphate buffer. The aliquots of rat spinal cord membrane used in the assay are washed with two portions of cold Phosphate buffer followed by microcentrifugation at 4° C., at 14,000 rpm between washings. The final pellets are then resuspended in a volume of phosphate buffer to provide concentrations appropriate to the assay conditions. The non-specific and total binding are defined by 10 mM final concentration of glycine and phosphate buffer only, respectively.

The reaction mixture is prepared by combining 150 μg of the rat spinal cord membrane with [3H]-strychnine to a final concentration of 7 nM and glycine or test compound. The reaction mixture is incubated for two hours while shaking on ice. The plates are then harvested onto GFC filters using a 48 w Brandall Harvestor. The GFC filter is pre-treated for at least 30 minutes with a solution of 0.5% BSA made is distilled water to reduce non-specific binding. The plate wells are washed with 4-5 volumes of cold phosphate buffer. The filters are then transferred to scintillation vials and 2 mls of scintillant is added to each vial. The vials are allowed to sit overnight before being counted in a Beckman β-counter. The data is analyzed using Prism software.

The compounds of the present invention show no significant binding to the glycine receptor.

We claim:

1. A compound of Formula 1: 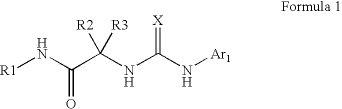
wherein:
R₁ is selected from cycloalkyl, heterocycloalkyl, aryl and heteroaryl
wherein R₁ is optionally substituted with one or more substituents Rₐ, wherein Rₐ is independently selected from the group consisting of alkyl, halo, haloalkyl, nitro, alkenyl, alkynyl, alkoxy, −(R₇)ₙNR₈R₉ (wherein R₇ is selected from alkyl, alkoxy, and oxyalkyl, R₈ and R₉ can be independently selected from H, and alkyl, or R₈ and R₉ can join together such that NR₈R₉ form a 5 or 6-member heterocyclic ring, and n is selected from 0 and 1), and the substituent Rₐ is optionally further substituted with one or more substituents selected from the group consisting of alkyl, alkoxy, halo, cyano, alkanoyl, haloalkyl, thioalkyl and nitro, —(R₇)ₙNR₈R₉, wherein R₇, R₈, R₉, and n are as defined above.
R₂ and R₃ are:
a) independently selected from the group consisting of H, alkyl, haloalkyl, aralkyl optionally substituted aryl, optionally substituted heteroaryl and optionally substituted, saturated or unsaturated, 5-or 6-membered, homocyclic or heterocyclic rings wherein the optional substituent may be selected from the group consisting of H, alkyl, alkoxy, and halo;
or
b) join together to form a 3, 4, 5, 6 or 7 member spirocyclic ring;
X is slelected from O, S, NH and NCN;
Ar₁ is phenyl and is optionally substituted with one or more substituents R_{b},
wherein the substituent(s) R_{b} are independently selected from the group consisting of alkyl, alkoxy, nitro halo, haloalkoxy, —(R₇)ₙNR₈R₉, —S(O)₂NR₁₀R₁₁ and —O—(CH₂)ₘNR₁₀R₁₁ (wherein R₇ is selected from alkyl, alkoxy, and oxyalkyl, R₈ and R₉ can be independently selected from H, and alkyl, or R₈ and R₉ can join together such that NR₈R₉ form a 5 or 6-member heterocyclic ring, and n is selected from 0, 1, 2, 3, 4 and 5 and R₁₀ and R₁₁ are independently selected from H, or alkyl, or R₁₀ and R₁₁ can join together such that NR₁₀R₁₁to form a 5 or 6-member heterocyclic ring and m is selected from 1, 2, 3, 4 and 5) and;
the substituent R_{b} is optionally further substituted with one or more substituents selected from the group consisting of alkyl, alkoxy, halo, cyano, alkanoyl, haloalkyl, thioalkyl, nitro, —(R₇)ₙNR₈R₉ wherein R₇, R₈, R₉ and n are as described above,
with the proviso that Ar₁ does not have a substituent at the 2-position selected from the following groups, nitro, haloalkyl, cyano, —C(O)R₁₂ —C(O)OR₁₂, —C(O)NR₁₂R₁₃, —S(O)R₁₂, —S(O)₂R₁₂, and —S(O)₂NR₁₂R₁₃ (wherein R₁₂ and R₁₃ are independently selected from H and alkyl), and,
the second proviso that Ar₁ does not have an alkanoyl substituent at the 4 position,
and a salt solvate or hydrate thereof.

2. A compound of claim 1 wherein Ar₁ is substituted with one or more substituents, Rₐ, wherein the substituent(s) Rₐ are selected from the group consisting of alkyl, alkoxy, nitro, acetyl, halo, haloalkyl, —S(O)₂NR₁₀R₁₁, —O-(CH₂)ₙNR₁₀R₁₁, wherein R₁₀ and R₁₁ are independently selected from H, or alkyl, or R₁₀ and R₁₁ can join together such that NR₁₀R₁₁ form a 5 or 6 member heterocyclic ring.

3. A compound of claim 2 wherein there are two substituents R₆, independently selected from the group consisting of nitro, methoxy, and ethoxy.

4. A compound of claim 3 wherein the two substituents R₆ are a nitro substituent at the 5-position and a methoxy substituent at the 2-position.

5. A compound as defined in claim 1 wherein R₁ is optionally substituted and is selected from the group consisting of phenyl, naphthyl, tetrahydro-naphthyl, indanyl, quinolinyl and pyridyl.

6. A compound of claim 5 wherein R₁ is indanyl.

7. A compound of claim 5 wherein R₁ is optionally substituted pyridyl wherein the substituent(s) Rₐ are selected from the group consisting of alkyl, and haloalkyl.

8. A compound of claim 5 wherein R₁ is optionally substituted phenyl wherein the substituent(s) Rₐ are selected from the group consisting of alkyl, halo, haloalkyl, nitro, vinyl, alkoxy, —(R₇)ₙNR₈R₉ wherein R₇ is selected from alkyl, alkoxy, and oxyalkyl, R₈ and R₉ can be independently selected from H, and alkyl, or R₈ and R₉ can join together such that NR₈R₉ form a heterocyclic ring, and n is selected from 0 and 1.

9. A compound of claim 8 wherein R₁ is selected from mono or di-substituted phenyl with the substituents selected independently from the group consisting of alkyl, halo and haloalkyl.

10. A compound as defined in claim 1 wherein R₂ and R₃ are independently selected from, H, alkyl, aralkyl, optionally substituted aryl, optionally substituted heteroaryl and optionally substituted saturated or unsaturated 5 or 6-membered homocyclic, or heterocyclic rings.

11. A compound as defined in claim 10 wherein R₂ and R₃ are selected independently from H, phenyl, 3-thiophene, sec-butyl, 3,4-difluorophenyl, cyclohexyl, 3-trifuoromethylphenyl, t-butyl, isopropyl, methyl, benzyl, trifuoromethyl.

12. A compound as defined in claim 10 wherein R₂ and R₃ together form a 3, 5 or 6 member spirocycle.

13. A compound of claim 1 selected from the group consisting of:
2-[3-(2-methoxy-5-nitro-phenyl)-thioureido]-N-(2-indanyl)-2-(3-thienyl) acetamide E42.2;
2-[3-(2-methoxy-5-nitro-phenyl)-thioureido]-N-(3,4-dimethylphenyl)-2-phenyl acetamide E32.2;
2-[3-(2-methoxy-5-nitro-phenyl)-ureido]-N-(3,4-dimethylphenyl)-2-phenyl acetamide E32.5;
(R)-2-[3-(2-methoxy-5-nitro-phenyl)-thioureido]-N-(3,4-dimethylphenyl)-2-phenyl acetamide E33.1*;
2-[3-(2-methoxy-5-nitro-phenyl)-ureido]-N-(2-indanyl)-2-(3-thienyl) acetamide E42.1;
(R)-2-[3-(2-nitro -5-methoxy-phenyl)-ureido]-N-(2-indanyl)-2-phenyl acetamide E29.1 *;
(R)-2-[3-(2-nitro-5-methoxy-phenyl)-ureido]-N-(4-chlorophenyl)-2-phenyl acetamide E4.1; and
(R)-2-[3-(2-methoxy-5-nitro-phenyl)-ureido]-N-(3-trifluromethylphenyl)-2-phenyl acetamide E31.2.

14. A pharmaceutical composition comprising a therapeutically effective amount of a compound of claim 1 and a pharmaceutically acceptable carrier.

15. A method for treating a patient having a medical condition for which a glycine transport inhibitor is indicated, comprising the step of administering to a patient a pharmaceutical composition as described in claim 14.

16. A method according to claim 15 wherein the medical condition is schizophrenia, cognitive dysfunction, or Alzheimer's disease.
